(19) United States
(12) Patent Application Publication
(10) Pub. No.: US 2024/0269150 A1
(43) Pub. Date: Aug. 15, 2024

(54) WOUND TREATMENT COMPOSITION

(71) Applicant: CS MEDICA A/S, Copenhagen (DK)

(72) Inventor: Lone HENRIKSEN, Fredensborg (DK)

(21) Appl. No.: 18/289,540

(22) PCT Filed: May 5, 2022
(86) PCT No.: PCT/EP2022/062150

§ 371 Date: Nov. 3, 2023

(30) Foreign Application Priority Data
May 6, 2021 (DK) PA 2021 70213

(51) Int. Cl. A61K 31/00; A61K 9/00; A61K 9/06; A61K 47/10; A61K 47/18; A61K 47/32; A61K 47/36; A61P 17/02

(52) U.S. Cl. CPC A61K 31/658; A61K 9/0014; A61K 9/06; A61K 47/10; A61K 47/186; A61K 47/32; A61K 47/36; A61P 17/02

(57) ABSTRACT

Wound treatment compositions and their use in the treatment and/or alleviation of wounds or sores are disclosed. The compositions comprise 0.1-5% cannabidiol (CBD), 25-85% water and one or more penetrator(s), and optionally one or more further agents, such as gelators, emollients, wound healing compounds, anti-microbial agents, pH-stabilizers and/or chelating agents.

### FIELD OF THE INVENTION

The present invention relates to a wound treatment composition for use in the treatment and/or alleviation of wounds and/or sores in a subject. Treatments may comprise application of a cannabinoid-comprising composition, in particular a cannabidiol-comprising composition.

### BACKGROUND OF THE INVENTION

Cannabinoids such as cannabidiol (CBD; CAS no. 3956-29-1) have been associated with wound healing, pain relief and anti-inflammatory effects.

WO 2020/263643 “Transdermal formulations” pertains to transdermal formulations comprising 0.05% to 50% w/w of an active agent and a pharmaceutically acceptable carrier.

WO2019178360 “Transdermal and/or dermal delivery of lipophilic active agents” concerns a dermal gel composition for wound healing comprising a lipophilic active agent.

WO2020024056 “Compositions comprising cannabinoids and absorbable material and uses thereof” relates to topical compositions suitable for topical administration.

Bruni et al 2018 (Molecules, 23(10): 2478) pertains to cannabinoid delivery systems for pain and inflammation treatment.

There is a need for compositions for efficient wound- and/or sore-treatment.

### SUMMARY OF THE INVENTION

As presented herein, surprisingly and/or unexpectedly, and from a wide range of component candidates and concentration ranges, the inventor has found the following compositions to be effective in relation to wound treatment and/or wound care concerning skin wounds in a subject.

In a first aspect, the present invention concerns a composition, such as a wound treatment composition comprising:
a) 0.1-5%, 0.2-2%, 0.3-1%, 0.4-0.75%, or around 0.5% (w/w) cannabidiol (CBD);
b) 25-85%, 35-75%, 45-70% or 55-65% (w/w) water; and
c) One or more penetrator(s) and/or penetration enhancer(s) such as propylene glycol and/or pentylene glycol;
and optionally one or more of:
   i. One or more gelator(s), such as polyacrylic acid/polyacrylate, e.g. 2-propeonic acid homopolymer and/or Carbomer;
   ii. One or more skin moisturizer(s) and/or skin conditioner(s), such as panthenol and/or allantoin;
   iii. One or more further wound healing compound(s), such as hyaluronic acid and/or its salt;
   iv. One or more anti-microbial agent(s), such as benzalkonium chloride; and/or
   v. One or more pH stabilizer(s) and/or buffering agent(s); such as aminomethyl propanol (AMP),
   vi. One or more chelating agent(s), such as phytic acid and/or its salt;
      including any combination(s) of (i)-(vi).

The composition, such as a wound treatment composition, can be formulated for topical application, such as a gel. Such gels usually comprise polyacrylic acid based gelator, such as Carbomer or sodium polyacrylate/polyacrylic acid.

In a second aspect, the present invention relates to a method for providing a composition, such as a wound treatment composition, such as a composition according to the first aspect, said method comprising:
a) providing CBD in solid form, preferably crystalline form and/or with a purity of at least 95% (w/w), 98% (w/w), 99% (w/w), 99.5% (w/w), 99.8% (w/w) or more than 98% (w/w);
b) dissolving the CBD from step (a) in a penetrator and/or penetration enhancer, such as propylene glycol;
c) providing an aqueous gel, such as by dissolving hyaluronic acid and/or its salt in water;
d) combining the dissolved CBD from step (b) with the aqueous gel from step (c); and
e) addition of the remaining ingredients to provide the wound treatment composition.

In a third aspect, the present invention pertains to a composition, such as a wound treatment composition provided by a method according to the second aspect.

In a fourth aspect, the present invention concerns a receptacle comprising a composition, such as a wound treatment composition according to the first, third or seventh aspect.

In a fifth aspect, the present invention relates to a kit comprising a receptacle according to the fourth aspect, and optionally, comprising an instruction for use.

In a sixth aspect, the present invention pertains to a method for treatment of a wound or sore of a subject, comprising topical application of a composition according to the first, third or seventh aspect.

In a seventh aspect, the present invention concerns a composition according to the first or third aspect for use as a medicament, or cosmetics. This may comprise treatment of a skin wound, sore, condition and/or disease, such as one or more of wounds, sore, conditions and/or diseases as disclosed herein.

In an eighth aspect, the present invention concerns a CBD-comprising composition, such as a topical composition, wherein the CBD used in the formulation is crystalline. In some embodiments, the CBD is of type A (needle-like crystals) or capable of forming needle-like crystals.

In a ninth aspect, the present invention pertains to a dosage regimen, comprising administering a topical composition, in particular CBD-comprising topical composition as disclosed herein. In some embodiments, the CBD is of “type A”.

### DESCRIPTION OF THE DRAWINGS/FIGURES

FIG. 1: microscope picture of cannabinol (CBD) forming needle-like crystals. The CBD crystals were sourced from www.enecta.com.

FIG. 2: microscope picture of cannabinol (CBD) forming cluster- or bunch-like crystals. The CBD crystals were sourced from www.pharma-hemp.com.

### DETAILED DESCRIPTION OF THE INVENTION

### Definitions

In the context of the present invention, the singular form of a word may include the plural, and vice versa, unless the context clearly dictates otherwise. Thus, the references “a,” “an” and “the” are generally inclusive of the plurals of the respective terms. For example, reference to “an ingredient” or “a method” may include a plurality of such “ingredients” or “methods.”

Similarly, the words “comprise,” “comprises,” and “comprising” are to be interpreted inclusively rather than exclusively. Embodiments provided by the present disclosure may lack any element that is not specifically disclosed herein. Thus, a disclosure of an embodiment defined using the term “comprising” is also a disclosure of embodiments “consisting essentially of” and “consisting of the disclosed components”. Thus, the term “comprising” is generally to be interpreted as specifying the presence of the stated parts, steps, features, or components, but does not exclude the presence of one or more additional parts, steps, features, or components. For example, a composition comprising a chemical compound may thus comprise additional chemical compounds.

Generally, compositions as disclosed herein, in particular topical compositions such as wound and/or sore treatment compositions may comprise one or more pharmaceutically acceptable adjuvant(s), such as pharmaceutically acceptable carrier(s), excipient(s), stabilizer(s), salt(s) or buffer(s) or the like.

Where used herein, terms like “for example”, “e.g.” or “such as”, particularly when followed by a listing of terms, is merely exemplary and illustrative, and should not be deemed to be exclusive or comprehensive. Any embodiment disclosed herein may be combined with any other embodiment disclosed herein.

Unless expressed otherwise, all percentages expressed herein are by weight of the total weight of the composition. Thus, unless indicated otherwise, “%” indicates “% weight/weight (w/w)”, also called “weight %” or “% by weight”.

In the context of the present invention, the terms “about”, “around”, “approximately” or the symbol “˜” can be used interchangeably, and are meant to comprise variations and/or uncertainties generally accepted in the field, e.g. comprising analytical errors and the like. Thus “about” may also indicate measuring uncertainty commonly experienced in the art, which can be in the order of magnitude of e.g. +/−1, 2, 5, 10, or even 20 percent (%). Furthermore, “about” may be understood to refer to numbers in a range of numerals, for example the range of +/−20, +/−15, +/−10, +/−5, +/−2, +/−1, +/−0.5, +/−0.1% of the referenced number. Moreover, all numerical ranges herein should be understood to include all integers, whole or fractions, within the range.

As used herein, the term “in some embodiments” is meant to comprise “in one embodiment”, “in some embodiments”, and “in one or more embodiments”.

In the context of the present invention, the terms “subject” or “patient” can be used interchangeably, and are meant to comprise a human, animal and/or mammal. In particular, a human subject can e.g. be selected from one or more of: female, male, senior, adult, adolescent, child, or infant. An animal subject can e.g. be selected from pet, husbandry, mammal, reptile, bird, and/or animal in a zoo.

In the context of the present invention, the term “treatment” is meant as an act aiming at alleviating, lessen, improving and/or curing any symptom(s), condition(s), or disease(s) in a subject. The effect of the treatment may also comprise reduction in pain and/or discomfort. A treatment may also result in a faster recovery and/or healing compared to a control. A further effect of a treatment may also comprise a recovery/healing with less complications compared to a control. A control can e.g. be no treatment or treatment with a placebo. Generally, a “treatment” in the present context comprises topical application of a suitable amount of a wound healing composition onto and/or around the wound, usually several times per day, as further elucidated herein.

“Skin” can be described as the layer of usually soft, flexible outer tissue covering the body of an animal, in particular vertebrate animal. The three main functions of the skin are believed to be protection, regulation, and sensation. The skin is believed to comprise three layers, the (i) epidermis, (ii) dermis and (iii) hypodermis, also called subcutaneous tissue. A “wound” or “sore” may concern one or more of these layers.

The epidermis can be further subdivided into the following strata or layers (beginning with the outermost layer): stratum corneum, stratum lucidum (only in palms and soles), stratum granulosum, stratum spinosum and stratum basale (also called the stratum germinativum).

In the context of the present invention, a “wound” refers to a type of injury which often, but not necessarily happens relatively quickly in which skin is burned, frozen, torn, cut, punctured (an open wound), and/or or where blunt force trauma causes a contusion (a closed wound). A “wound” may also refer to an injury which damages the epidermis of the skin. A “wound” can e.g. be: (i) a “clean wound”, such as a wound made under sterile conditions, where there are no or very few organisms present, and the skin is likely to heal without complications; a (ii) “contaminated wound”—usually resulting from accidental injury, and where there are pathogenic organisms and often foreign bodies in the wound; (iii) an “infected wound”—the wound has pathogenic organisms present and multiplying and usually exhibiting clinical signs of infection, such as yellow appearance, soreness, redness, oozing pus and the like; or (iv) a “colonized wound”—such as a chronic situation, containing pathogenic organisms, difficult to heal, e.g. bedsore.

In the context of the present invention, a wound caused by freezing can also be called “frost bite”.

In some embodiments, a “wound” can be the result of a medical and/or cosmetical treatment and/or intervention, such as surgery or tattooing.

Furthermore, a wound can be caused intentionally (e.g. surgery), or accidentally.

The term “wound” may also comprise:

Incisions or incised wounds—often caused by a clean, sharp-edged object such as a knife, razor, or glass splinter; lacerations—irregular tear-like wounds caused by some blunt trauma. Lacerations and incisions may appear linear (regular) or stellate (irregular); abrasions or grazes—superficial wounds in which the topmost layer of the skin (the epidermis) is scraped off. Abrasions are often caused by a sliding fall onto a rough surface such as asphalt, tree bark or concrete; avulsions—injuries in which a body structure is forcibly detached from its normal point of insertion. A type of amputation where the extremity is pulled off rather than cut off. When used in reference to skin avulsions, the term ‘degloving’ is also sometimes used as a synonym; puncture wounds—caused by an object puncturing the skin, such as a splinter, nail or needle; penetration wounds—caused by an object such as a knife entering and coming out from the skin; and gunshot wounds—caused by a bullet or similar projectile driving into or through the body. There may be two wounds, one at the site of entry and one at the site of exit, generally referred to as a “through-and-through.”

Burn wounds—A “burn wound” or “burn” is a type of injury to skin, or other tissues, caused by heat, cold, electricity, chemicals (e.g. corrosive chemicals), friction, or radiation (e.g. sunlight and/or UV light). Most burns are due to heat from hot liquids, solids, fire or chemicals. Burns that affect only the superficial skin layers are known as superficial or first-degree burns. They appear red without blisters and pain and typically lasts around three days. When the injury extends into some of the underlying skin layer, it is a partial-thickness or second-degree burn. Blisters are frequently present, and they are often very painful. Healing can require up to eight weeks and scarring may occur. In a full-thickness or third-degree burn, the injury extends to all layers of the skin. Often there is no pain and the burnt area is stiff. Healing typically does not occur on its own. A fourth degree burn additionally involves injury to deeper tissues, such as muscle, tendons, or bone. The burn is often black and frequently leads to loss of the burned part.

Frost bite(s)—Such wound(s) can be caused by freezing, such as wounds caused by exposure of skin/body parts to low temperatures, causing freezing of skin and/or other tissues. Usually, such wounds can e.g. be caused by cold air, usually in combination with inadequate protective clothing, contact with liquid gas (e.g. CO₂, N₂, or the like), or contact with frozen solid matter, such as metal surfaces or the like. These can be classified and/or described as follows: First degree frostbite is superficial, surface skin damage that is usually not permanent. Early on, the primary symptom is loss of feeling in the skin. In the affected areas, the skin is numb, and possibly swollen, with a reddened border. In the weeks after injury, the skin's surface may slough off. In second degree frostbite, the skin develops clear blisters early on, and the skin's surface hardens. In the weeks after injury, this hardened, blistered skin dries, blackens, and peels. At this stage, lasting cold sensitivity and numbness can develop. In third-degree frostbite, the layers of tissue below the skin freeze. Symptoms include blood blisters and “blue-grey discoloration of the skin”. In the weeks after injury, pain persists, and a blackened crust (eschar) develops. There can be long-term ulceration and damage to growth plates. In fourth degree frostbite, structures below the skin are involved like muscles, tendon, and bone. Early symptoms include a colourless appearance of the skin, a hard texture, and painless rewarming. Later, the skin becomes black and mummified. The amount of permanent damage can take one month or more to determine.

A “wound” may also be described as a “sore”, in particular a skin-related sore. A “sore” can e.g. be described as a term for a usually painful, discomfortable and/or irritating lesion of the skin or mucous membranes. A “sore” may e.g. comprise bay sore (chiclero ulcer); bed sore (decubitus ulcer); canker sore (recurrent aphthous stomatitis); chrome sore (chrome ulcer); cold sore (herpes febrilis), desert sore (a type of phagedenic ulcer seen in South Africa and Australia with initial papulovesicular lesions on the limbs, which later rupture and form painful ulcers; it probably represents an infected ulcer from some previously neglected lesion); pressure sore (decubitus ulcer); saddle sore; soft sore (chancroid); summer sore (cutaneous habronerniasis); veldt sore (desert sore); veneral sore (any sore that accompanies or manifests a venereal disease).

The terms “wound” and “sore” can thus be overlapping and may also be used interchangeably. Apart from “sore”, the term “wound” may also comprise any one of “ulcer”, “blister”, and/or “rash”. In the context of the present invention, the term “wound treatment composition” is meant to comprise “sore treatment composition”.

In the context of the present invention, the term “ulcer” is meant to comprise a sore on the skin or a mucous membrane, accompanied by the disintegration of tissue. Ulcers can result in complete loss of the epidermis and often portions of the dermis and even subcutaneous fat. Ulcers are most common on the skin of the lower extremities and in the gastrointestinal tract. An ulcer that appears on the skin is often visible as an inflamed tissue with an area of reddened skin. A skin ulcer is often visible in the event of exposure to heat or cold, irritation, or a problem with blood circulation. Ulcers can also be caused due to a lack of mobility, which causes prolonged pressure on the tissues. This stress in the blood circulation is transformed to a skin ulcer, commonly known as bedsores or decubitus ulcers. Ulcers often become infected, and pus forms.

Ulcers can be caused by a variety of factors, but the main cause appears to be impaired blood circulation. Especially, chronic wounds and ulcers are caused by poor circulation, either through cardiovascular issues or external pressure from a bed or a wheelchair. A very common and dangerous type of skin ulcers are caused by what are called pressure-sensitive sores, more commonly called bed sores and which are frequent in people who are bedridden or who use wheelchairs for long periods. Other causes producing skin ulcers include bacterial or viral infections, fungal infections and cancers. Blood disorders and chronic wounds can result in skin ulcers as well. Venous leg ulcers due to impaired circulation or a blood flow disorder are more common in the elderly. Rare causes of skin ulcers include pyoderma gangracnosum, lesions caused by Crohn's disease or ulcerative colitis, granulomatosis with polyangiitis, morbus Behçet, and infections that are usually seen in those who are immunocompromised, for example ecthyma gangraenosum.

In the context of the present invention, a “rash” is meant to comprise a change of the human skin which affects its colour, appearance, or texture. A rash may be localized in one part of the body or affect all the skin. Rashes may cause the skin to change colour, itch, become warm, bumpy, chapped, dry, cracked or blistered, swell, and may be painful. The causes for rashes may vary widely, such as: medication side effects; anxiety; allergies, for example to food, dyes, medicines, vaccinations, insect stings, metals such as zinc or nickel; such rashes are often called hives; skin contact with an irritant; fungal infection (e.g.) ringworm; skin diseases such as eczema or acne; exposure to sun (sunburn) or heat; friction due to chafing of the skin; irritation such as caused by abrasives impregnated in clothing rubbing the skin; secondary syphilis; and/or poor personal hygiene. Less common causes for rashes may comprise: autoimmune disorders such as psoriasis; lead poisoning; pregnancy; repeated scratching on a particular spot; Lyme disease or Lyme borreliosis, an infectious disease caused by the Borrelia bacterium usually spread by ticks; scarlet fever; and/or COVID-19.

In the context of the present invention, a “blister” is meant to comprise a usually small pocket of body fluid (lymph, serum, plasma, blood, or pus) within the upper layers of the skin, usually caused by forceful rubbing (friction), burning, freezing, chemical exposure or infection. Most blisters are filled with a clear fluid, either serum or plasma. However, blisters can be filled with blood (known as “blood blisters”) or with pus (for instance, if they become infected). The terms “vesicle” and “bulla” can also be used, and usually refer to blisters of smaller or greater size, respectively. A blister may form when the skin has been damaged by friction or rubbing, heat, cold or chemical exposure. Fluid collects between the upper layers of skin (the epidermis) and the layers below (the dermis). This fluid cushions the tissue underneath, protecting it from further damage and allowing it to heal.

Friction blisters can be caused by intense rubbing, as may any friction on the skin if continued long enough. This kind of blister is most common after walking long distances or by wearing old or poorly fitting shoes. Blisters are most common on the hands and feet, as these extremities are susceptible while walking, running, or performing repetitive motions, such as joystick manipulation whilst playing certain video games, digging with a shovel, playing guitar or bass, etc. Blisters form more easily on damp skin than on dry or soaked skin and are more common in warm conditions. Less-aggressive rubbing over long periods of time may cause calluses to form rather than a blister. Both blisters and calluses can lead to more serious complications, such as foot ulceration and infection, particularly when sensation or circulation is impaired, as in the case of diabetes, neuropathy or peripheral artery disease (PAD).

Burning, in particular first- and second-degree burns may result in blistered skin; however, it is characteristic of second-degree burns to blister immediately, whereas first-degree burns can have blisters after a couple of days. Sunburn or other forms of radiation can also result in blisters.

Blisters can also form on the hands and feet as a result of tissue damage incurred by frostbite.

Chemical exposure may also cause blisters. Sometimes, the skin will blister when it comes into contact with a cosmetic, detergent, solvent, or other chemicals such as nickel sulphate, balsam of Peru, or urushiol (poison ivy, poison oak, poison sumac). This may also be known as contact dermatitis. Blisters can also develop as a result of an allergic reaction to an insect bite or sting. Some chemical warfare agents, known as blister agents or vesicants, cause large, painful blisters wherever they contact skin, such as mustard gas.

A blood blister usually forms when a minute blood vessel close to the surface of the skin ruptures (breaks), and blood leaks into a tear between the layers of skin. This can happen if the skin is crushed, pinched or aggressively squeezed.

There are also a number of medical conditions that cause blisters. The most common are chickenpox, herpes, impetigo, and a form of eczema called dyshidrosis. Other, usually rarer conditions that cause blisters may comprise: Bullous pemphigoid, a skin disease that causes large, tightly filled blisters to develop, usually affecting people over the age of 60; Pemphigus, a serious skin disease in which blisters develop if pressure is applied to the skin. These blisters burst easily, leaving raw areas that may become infected; Dermatitis herpetiformis, a skin disease that causes intensely itchy blisters, usually on the elbows, knees, back and buttocks. The blisters usually develop in patches of the same shape and size on both sides of the body; Chronic bullous dermatosis, a disease that causes clusters of blisters on the face, mouth or genitals; Cutaneous radiation syndrome; and Epidermolysis bullosa, a group of rare medical conditions that result in easy blistering of the skin and mucous membranes. Blisters occur with minor trauma or friction and are painful. Its severity can range from mild to fatal. Those with mild cases may not develop symptoms until they start to crawl or walk. Complications may include esophageal narrowing, squamous cell skin cancer, and the need for amputations.

Friction blisters are caused by excess shear stress between the bottom and surface of the skin and the body. The strata of skin around the stratum spinosum are most susceptible to shear. As the stratum spinosum tears away from the connecting tissues below, plasma from the cells diffuses out. This plasma solution helps new cells divide and grow into new connective tissues and epidermal layers. The clear fluid will be reabsorbed as new cells develop and the swollen appearance will subside. Painful blisters located on hands (palmar surface) and feet (plantar surface) are due to tissue shearing deeper in the epidermis, near nerve endings. Lower tissues are more susceptible to infection.

In a first aspect, the present invention concerns a composition, such as a wound and/or sore treatment composition comprising CBD, and a penetrator and/or penetration enhancer. Usually, the composition is formulated for topical application, such as, but not limited to, a gel. Such a composition can also be a wound-care composition and be used thereto.

In some embodiments, such a wound treatment composition may comprise:
a) 0.1-5%, 0.2-2%, 0.3-1%, 0.4-0.75%, or around 0.5% (w/w) cannabidiol (CBD);
b) 25-85%, 35-75%, 45-70% or 55-65% (w/w) water; and
c) one or more penetrator(s) and/or penetration enhancer(s) such as propylene glycol and/or pentylene glycol.

Cannabidiol, CAS no. 3956-29-1 is a non-psychoactive cannabinoid. It can be provided in different purities, and is usually extracted from Cannabis sativa by methods known in the art. In the context of the present invention, CBD with a high degree of purity is generally preferred, such as “crystalline” CBD, comprising neither oil nor further cannabinoids, such as psychoactive or non-psychoactive cannabinoids in significant amounts.

In particular, when absence of oil(s) and/or fat(s) is desired, common sources of CBD, such as CBD-comprising oils are not desirable. Thus, in some embodiment, CBD is provided in essentially pure form, such as in crystalline or powder form and/or with a purity of 95%, 98%, 99%, 99.5%, 99.8% or more than 99.8%. Without wanting to be bound by any theory, it is believed that the use of CBD in crystalline may further contribute in a positive fashion, such as that less CBD is required to provide a similar effect compared to a crude CBD preparation. This is surprising, as according to general belief, further cannabinoids present in such crude CBD preparations are believed to provide a synergistic effect.

Generally, the water used in the formulations is of drinking water quality. It may also be distilled or deionized water, such as “MilliQ water”.

CBD-comprising compositions described herein usually comprise one or more skin penetrating enhancer(s), such as a mixture of two or more skin penetration enhancers. Commonly, a “skin penetrating enhancer”, “penetrating enhancer” or “penetrator”—all three terms can be used interchangeably herein—improves the ability of one or more relevant component(s)/ingredient(s) of the composition, such as CBD to pass through the epidermal layer and the dermal layer of the skin to reach the adipose tissue that underlies the skin wherein adipocytes are increased in number and/or size. Without wanting to be bound by any theory, it is believed that this may be accomplished by a number of different mechanisms including, for example, by extracting lipids from the stratum corneum, increasing the partitioning of the active ingredients into the skin, and disrupting the lipid bilayer of the stratum corneum, thus rendering the stratum corneum structure more fluid and increasing the ability of the composition including the cannabinoids to diffuse through the stratum corneum.

In some embodiments, the “penetrator enhancer” is provided in a concentration of 0.1-15% (w/w), 0.5-12% (w/w), 1.0-10% (w/w), 2.0-7.5% (w/w), 4.0-6.0% (w/w), or around 5% (w/w). Suitable skin penetrating enhancers can be, for example, sulfoxides, alcohols, fatty acids, fatty acid esters, polyols, amides, surfactants, terpenes, alkanones, and organic acids, among others. Specific examples of suitable sulfoxides include dimethylsulfoxide (DMSO) and decylmethylsulfoxide, among others. Suitable alcohols include alkanols such as ethanol, propanol, butanol, pentanol, hexanol, octanol, n-octanol, nonanol, decanol, 2-butanol, 2-pentanol, and benzyl alcohol; fatty alcohols, such as caprylic alcohol, decyl alcohol, lauryl alcohol, 2-lauryl alcohol, myristyl alcohol, cetyl alcohol, stearyl alcohol, oleyl alcohol, linoleyl alcohol, and linolenyl alcohol; and isopropyl alcohol. Examples of suitable fatty acids include linear fatty acids such as valeric acid, heptanoic acid, pelagonic acid, caproic acid, capric acid, lauric acid, myristic acid, stearic acid, oleic acid, and caprylic acid; and branched fatty acids, such as isovaleric acid, neopentanoic acid, neoheptanoic acid, neononanoic acid, trimethyl hexanoic acid, neodecanoic add, and isostearic acid. Examples of suitable fatty acid esters include aliphatic fatty acid esters such as isopropyl n-butyrate, isopropyl n-hexanoate, isopropyl n-decanoate, isopropyl myristate, isopropyl palmitate, and octyldodecyl myristate; alkyl fatty acid esters such as ethyl acetate, butyl acetate, methyl acetate, methylvalerate, methylpropionate, diethyl sebacate, and ethyl oleate; and diisopropyl adipate and dimethyl isosorbide. Examples of suitable polyols include propylene glycol, butylene glycol, polyethylene glycol, ethylene glycol, diethylene glycol, triethylene glycol, dipropylene glycol, ethoxydiglycol, pentylene glycol, glycerol, propanediol, butanediol, pentanediol, hexanetriol, and glycerin. Examples of suitable amides include urea, dimethylacetamide, diethyltoluamide, dimethylformamide (DMF), dimethyloctamide, dimethyldecamide, biodegradable cyclic urea (e.g., 1-alkyl-4-imidazoline-2-one), pyrrolidone derivatives, biodegradable pyrrolidone derivatives (e.g., fatty acid esters of N-(2-hydroxyethyl)-2-pyrrolidone), cyclic amides, hexamethylenelauramide and its derivatives, diethanolamine, and triethanolamine. Examples of pyrrolidone derivatives include 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-methyl-4-methoxycarbonyl-2-pyrrolidone, 1-hexyl-4-meth-oxycarbonyl-2-pyrrolidone, 1-lauryl-4-methoxycarbonyl-2-pyrrolidone, N-cyclohexyl-pyrrolidone, N-dimethylaminopropylpyrrolidone, N-cocoalkypyrrolidone, N-tallowalkyl-pyrrolidone, and N-methylpyrrolidone. Examples of cyclic amides include 1-dodecylazacyclo-heptane-2-one (e.g., Azone), 1-geranylazacycloheptan-2-one, 1-famesylazacycloheptan-2-one, 1-geranylgeranylazacyclo-heptan-2-one, H3,7-dimethyloctyl) azacycloheptan-2-one, 1-(3,7,11-trimethyldodecyl)aza-cyclohaptane-2-one, 1-geranylazacyclohexane-2-one, 1-geranylazacyclopentan-2,5-dione, and 1-famesylazacyclopentan-2-one.

In some embodiments, the penetrator/penetration enhancer is or comprises a polyol. In some embodiments, the penetrator(s), such as a polyol, in particular a glycol is/are present in a concentration of 0.1-1.0% (w/w), 0.5-1.5% (w/w), 1.0-2.5% (w/w), 1.5-5.0% (w/w), 2.5-10% (w/w), or 5-15% (w/w). In some embodiments, the penetrator/penetration enhancer is or comprises a glycol. In some embodiments, the penetrator/penetration enhancer is or comprises propylene glycol. In some embodiments, the penetrator/penetration enhancer is or comprises pentylene glycol. In some embodiments, the penetrator/penetration enhancer is or comprises propylene- and pentylene glycol. In some embodiments, the one or more penetrator(s) and/or penetration enhancer(s) is or comprises propylene glycol, and/or pentylene glycol. In some embodiments, the penetrators comprise propylene glycol and/or pentylene glycol, and a further glycol.

In some embodiments, propylene glycol is provided in a concentration of 0.1-15% (w/w), 0.5-12% (w/w), 1.0-10% (w/w), 2.0-7.5% (w/w), 4.0-6.0% (w/w), or around 5% (w/w). In some embodiments, pentylene glycol is provided in a concentration of 0.1-15% (w/w), 0.5-12% (w/w), 1.0-10% (w/w), 1.5-7.5% (w/w), 2.0-6.0% (w/w), 2.5-4.0% (w/w), or around 5% (w/w). In some embodiments, the one or more penetrator(s) is/are selected from the list of penetrators disclosed herein. In some embodiments, the penetrator(s) is/are present in a concentration of 0.1-1.0% (w/w), 0.5-1.5% (w/w), 1.0-2.5% (w/w), 1.5-5.0% (w/w), 2.5-10% (w/w), or 5-15% (w/w). This can e.g. be a combination of two glycols, such as pentylene and propylene glycol.

In some embodiments, CBD can be dissolved in the penetrator, such as propylene glycol. This advantage can e.g. be exploited in the production process. Commonly, CBD is dissolved in oil or alcohol. However, in some embodiments, oil and/or alcohol can be undesired, e.g. for the reasons discussed herein.

A composition, such as a wound and/or sore treatment composition disclosed herein may also comprise one or more pharmaceutically acceptable adjuvant(s). In some embodiments, the adjuvant can be selected from one or more of: antioxidant(s), emulsifier(s), pH regulating agent(s), such as acid(s), base(s) or salt(s) thereof, stabilizer(s), colorant(s), including any combinations thereof.

Often, the composition, such as the wound and/or sore treatment composition will comprise one or more further agents, such as one or more gelator(s), one or more emollient(s), one or more skin conditioner(s), one or more wound healing compound(s), one or more anti-microbial agent(s), one or more pH stabilizer(s); and/or one or more chelating agent(s), including any combination(s) thereof.

In some embodiments, the composition may thus comprise optionally one or more of (i)-(vi), such as:
i. One or more gelator(s), such as polyacrylic acid/polyacrylate, e.g. 2-propeonic acid, homopolymer and/or Carbomer);
ii. One or more skin moisturizer(s) and/or skin conditioner(s), such as panthenol and/or allantoin;
iii. One or more further wound healing compound(s), such as hyaluronic acid and/or its salt;
iv. One or more anti-microbial agent(s), such as benzalkonium chloride; and/or
v. One or more pH stabilizer(s) and/or buffering agent(s); such as aminomethyl propanol (AMP),
vi. One or more chelating agent(s), such as phytic acid and/or its salt;
   including any combination(s) of (i)-(vi).

The composition, such as the wound and/or sore treatment composition can be formulated for topical application, such as a gel. A wound treatment composition formulated as a gel provides e.g. the advantage of facilitating appropriate dosage and application, as well as one or more further advantages, as disclosed herein.

Thus, in some embodiments, the composition comprises one or more gelator(s) to provide a gel-like composition. A “gelator” is a substance or compound capable of forming a gel. Gels can e.g. be hydrogels, comprising a polymer or colloidal network. Examples of suitable gelators may comprise: 1-methyl-2,4-bis(N′-n-octadecylureido) benzene (MBB18), 1-methyl-2,4-bis(N′-n-dodecylureido) benzene (MBB12), bis(4′-stearamido phenyl) methane (BSM18), bis(4′-octanamido phenyl)methane (BOM8), 12-hydroxystearic acid, nucleobase, phenylalanine, d-glucosamine, RAD 16, EAK 16, RAD 16 I, RAD 16-II, KLD-12, Nucleopeptide (phenylalanine dipeptide link to a nucleobase), Guanosine derivatives, Carbomer, such as Carbomer 910, 934, 940, 941 and 934P (these numbers are an indication of molecular weight and the specific components of the polymer), IKVAV-Peptide amphiphiles, Heparin-binding peptide amphiphile LRKKLGKA-PA, A glycosylated amino acetate type of hydrogelator 1 C33O12N3H55, Gelator 4b (a derivative of d-gluconolactone) C16O7N2H24, Unimer U-15, Unimer U-151, Unimer U-1946, and/or Unimer U-6. In some embodiments, the gelator can be selected from one or more gelators as disclosed above or below.

In some embodiments, the one or more gelator(s) is provided in a concentration of 0.1-5%, 0.2-3% (w/w), 0.5-2% (w/w), 0.6-1.0% (w/w), or around 0.8% (w/w). In some embodiments, the gelator(s) is or comprises Carbomer, polyacrylic acid/polyacrylate, such as sodium polyacrylate. In some embodiments, the gelator is or comprises polyacrylic acid, polyacrylate, and/or 2-propeonic acid homopolymer. In some embodiments, the gelator is or comprises Carbomer. Poly(acrylic acid) (PAA; trade name Carbomer) is a synthetic high-molecular weight polymer of acrylic acid. The IUPAC name is poly(1-carboxyethylene). They may be homopolymers of acrylic acid, or crosslinked with an allyl ether of pentaerythritol, allyl ether of sucrose, or allyl ether of propylene. In a water solution at neutral pH, PAA is an anionic polymer, i.e. many of the side chains of PAA will lose their protons and acquire a negative charge. This makes PAAs polyelectrolytes, with the ability to absorb and retain water and swell to many times their original volume.

In some embodiments, hyaluronic acid/hyaluronate acts as a gelator, either alone, or in combination with a further gelator, such as polyacrylic acid/polyacrylate. Hyaluronic acid is a polymer of disaccharides, which are composed of D-glucuronic acid and N-acetyl-D-glucosamine, linked via alternating β-(1→4) and β-(1→3) glycosidic bonds. Hyaluronic acid can be 25,000 disaccharide repeats in length. Polymers of hyaluronic acid can range in size from 5,000 to 20,000,000 Da in vivo. The average molecular weight in human synovial fluid is 3-4 million Da, and hyaluronic acid purified from human umbilical cord is 3,140,000 Da; other sources mention average molecular weight of 7 million Da for synovial fluid. Hyaluronic acid combines with water and swells to form a gel. Furthermore, hyaluronic acid is believed to be involved in tissue regeneration and is used as a dermal filler for e.g. facial wrinkles, as hyaluronic acid is known to bind and absorb water up to 1000 times its own molecule weight. In some embodiments, hyaluronic acid/hyaluronate acts as gelator, in combination with a further gelator, such as Carbomer or the like.

The function of the gelator can be described as the provision of a gel or gel-like texture of the composition. Thereto, one or more thickeners or gelling agents can be provided. Such thickeners/gelling agent(s) can be selected from acrylate cross polymers, in particular C10-C30 alkyl acrylate cross polymers (such as commonly marketed under the tradename Carbopol®), hydroxyethyl cellulose, xanthan gum and/or any combinations thereof. The amount of gelator and/or thickener(s) can be considered sufficient when it ensures that the gel does not run off during application. In some embodiments, the gel may comprise a thickener and/or gelling agent selected from acrylate cross polymers, hydroxyethyl cellulose, xanthan gum and/or any combinations thereof.

In some embodiments, the composition comprises one or more skin moisturizer(s) and/or one or more skin conditioner(s).

Generally, the terms “moisturizer”, “skin moisturizer”, or “emollient” can be used interchangeably and are meant to comprise a cosmetic composition providing protection, moisturizing and/or lubrication of the skin. A moisturizer may also prevent dryness and irritation of the skin by moisturization. In the context of the present invention, the term “moisturizer” or “emollient” may also relate to the individual compound(s) that provide or improve such a moisturizing effect. Examples of such compounds may comprise: Panthenol, Allantoin, Isopropyl Myristate, pantothenic acid, Sodium Hyaluronate, Squalene, Phenoxyethanol, methyl-paraben, propyl-paraben, ethyl-paraben, butyl-paraben, lanolin, sorbitol, petrolatum, Stearic acid, Shea butter, Glyceryl stearate, Elastin, Hyaluronic acid, Olive oil, Glycerine Pharma 99.5% vegetable gum, rhizobian, and/or Sea Water.

In some embodiments, the emollient is or comprises panthenol. In some embodiments, the emollient is or comprises allantoin. In some embodiments, the emollient is or comprises panthenol and allantoin.

In some embodiments, hyaluronic acid/hyaluronate acts as gelator and/or emollient.

In some embodiments, the skin moisturizer is provided in a concentration of 0.1-5% (w/w), 0.15-3.0% (w/w), 0.2-1.5% (w/w), 0.5-0.8% (w/w), or around 0.65% (w/w). In some embodiments, the skin moisturizer(s) is or comprises panthenol. In some embodiments, the skin moisturizer(s) is or comprises allantoin. In some embodiments, the skin moisturizer(s) comprises panthenol and allantoin. In some embodiments, panthenol is provided in a concentration of 0.1-5% (w/w), 0.15-3.0% (w/w), 0.2-1.5% (w/w), 0.5-0.8% (w/w), or around 0.65% (w/w). In some embodiments, allantoin is provided in a concentration of 0.01-5% (w/w), 0.05-2% (w/w), 0.1-1% (w/w), 0.2-0.5% (w/w), or around 0.3% (w/w). In some embodiments, panthenol and allantoin is provided in a concentration of 0.01-5% (w/w), 0.05-2% (w/w), 0.1-1% (w/w), 0.2-0.5% (w/w), or around 0.3% (w/w) each. In some embodiments, panthenol and allantoin are provided in a combined concentration of 0.01-5% (w/w), 0.05-2% (w/w), 0.1-1% (w/w), 0.2-0.5% (w/w), or around 0.3% (w/w) each. In some embodiments, the emollient is selected from one or more of: Panthenol, Allantoin, Isopropyl Myristate, pantothenic acid, Sodium Hyaluronate, Squalene, Phenoxyethanol, methyl-paraben, propyl-paraben, ethyl-paraben, butyl-paraben, lanolin, sorbitol, petrolatum, Stearic acid, Shea butter, Glyceryl stearate, Elastin, Hyaluronic acid, Olive oil, Glycerine Pharma 99.5% vegetable gum, rhizobian, and/or Sea Water.

In some embodiments, the composition comprises a skin conditioner. In the context of the present invention, the term “skin conditioner” or “skin essence” is meant to comprise a component or composition providing softening of the skin. Often, a skin conditioner will also hydrate the skin, such as a moisturizer. In some embodiments, the skin conditioner is provided in a concentration of 0.1-5% (w/w), 0.15-3.0% (w/w), 0.2-1.5% (w/w), 0.5-0.8% (w/w), or around 0.65% (w/w). In some embodiments, the skin conditioner(s) is or comprises panthenol. In some embodiments, the skin conditioner(s) is or comprises allantoin. In some embodiments, the skin conditioner(s) comprises panthenol and allantoin. In some embodiments, allantoin is provided in a concentration of 0.1-5% (w/w), 0.15-3.0% (w/w), 0.2-1.5% (w/w), 0.5-0.8% (w/w), or around 0.65% (w/w). In some embodiments, panthenol is provided in a concentration of 0.1-5% (w/w), 0.15-3.0% (w/w), 0.2-1.5% (w/w), 0.5-0.8% (w/w), or around 0.65% (w/w). In some embodiments, allantoin and panthenol are provided in a combined concentration of 0.1-5% (w/w), 0.15-3.0% (w/w), 0.2-1.5% (w/w), 0.5-0.8% (w/w), or around 0.65% (w/w).

In some embodiments, the skin conditioner is selected from one or more of: Panthenol, Astrocaryum Vulgare Seed Butter, Astrocaryum Vulgare Seed Butter, Gossypium Hirsutum Seed Extract, Pentaclethra Macrophylla Seed Oil, Abies Alba Extract, Zanthoxylum Bungeanum Pericarp Extract, Zea Mays Germ Extract, Zymomonas Ferment Filtrate, Zingiber Officinale Root, Ziyu Glycoside II, Zostera Marina Callus Extract, Ulva Australis Extract, Actinidia Arguta Juice, Adenosine, Adonis Amurensis Extract, Aloe Barbadensis Leaf Extract, Amaranthus Spinosus Seed Oil, Ananas Sativus Fruit Juice, Black Soldier Fly Larva Oil, Azurite, Bacillus/Corchorus Olitorius Leaf Ferment Filtrate, Cajanus Cajan Leaf Extract, and/or Calcium Polyglutamate Crosspolymer.

In some embodiments, the skin conditioner may provide a further effect, such as a moisturizing effect.

In some embodiments, a skin conditioner may also act as an emollient, and vice versa. An example thereof is e.g. panthenol, which may act as skin conditioner and/or as emollient.

A wound and/or sore treatment composition may benefit from the presence of one or more further wound healing compound(s). In some embodiments, the composition comprises a wound healing compound. In the context of the present invention, a “wound healing compound” is a component that promotes wound healing and/or tissue regeneration. CBD is an example of a wound healing compound. In some embodiments, the further wound healing compound is, or comprises hyaluronic acid and/or its salt. Suitable concentrations for wound healing compound(s) may vary, such as around 0.1-5% (w/w). In some embodiments, the one or more further wound healing compound(s) is provided in a concentration of 0.1-5% (w/w), 0.2-3% (w/w), 0.3-1% (w/w), 0.35-0.75% (w/w), 0.4-0.6% (w/w), or around 0.5% (w/w). In some embodiments, the further wound healing compound is selected from one or more of: Honey (medical), hyaluronic acid, vitamin E, Aloe vera, Benzalkonium Chloride 0.13%, Propylene Glycol, Glycerin 20.0%, propolis, Petroleum jelly, curcumin, garlic, carbonoid oil, collagen, sorbitol, silver, Anethum graveolens, Anethum graveolens, Cinnamomum verum, Eucalyptus, Securigera securidaca, Trigonella foenum-graecum, Nelumbo nucifera, Neem leaf extracts, Chamomilla recutita, nitrofurazone, Bacl, Moltkia coerulea, and Allium sativum L. (Amaryllidaceae) including any combinations thereof.

In some embodiments, hyaluronic acid/hyaluronate acts as wound healing compound. In some embodiments, hyaluronic acid/hyaluronate acts as one or more of: gelator, emollient, and/or wound healing compound, including any combination(s) thereof.

The CBD-comprising compositions, such as the wound/sore treatment composition may further comprise a “preservative”. A preservative provides stability and/or increased stability of the composition, such as by preventing the growth of microbial organisms in the compositions, also called “anti-microbial agent” herein. In some embodiments, one or more suitable preservative(s) and/or anti-microbial agent(s) may e.g. be selected from: biocide, methylparabens, ethylparabens, propylparabens, butylparabens, organic acid, citric acid, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, piropylene glycol, benzaldehyde, butylated hydroxytoluene, butylated hydroxyanisole, formaldehyde donors, botanical extracts, monoglyceride, phenol, mercury components and any combination thereof. In some embodiments, one or more suitable anti-microbial agent(s) may be selected from one or more of: organic acid, salt of an organic acid, including any combination thereof. Without wanting to be bound by any theory, it is believed that CBD possesses an anti-microbial effect, probably comparable to some conventional antibiotics. CBD is believed to be active against pathogens, such as Staphylococcus aureus, Streptococcus pneumonie and/or Clostridioides difficile.

A composition, such as a wound and/or sore treatment composition may also benefit from the presence of one or more anti-microbial agent(s) or stabilizers, such as for storability and/or microbial safety of the product. In some embodiments, the anti-microbial agent is or comprises benzalkonium chloride. In some embodiments, the one or more anti-microbial agent(s) is provided in a concentration of 0.01-5% (w/w), 0.02-2% (w/w), 0.04-1% (w/w), 0.075-0.2% (w/w), or around 0.1% (w/w). In some embodiments, the anti-microbial agent is/are provided in a concentration of 0.01-2.5% (w/w), 0.025-0.1.0% (w/w), 0.05-0.5% (w/w), 0.075-0.2% (w/w), or around 0.1% (w/w). In some embodiments, the preservative may provide a further effect, such as pH-adjusting and/or buffering effect. In some embodiments, the anti-microbial agent(s) is/are selected from one or more anti-microbial agents/preservative(s) disclosed herein.

In some embodiments, a CBD-comprising composition is formulated such that a defined pH is provided. Generally, a neutral, near neutral, and/or slightly acidic pH, such as a pH mimicking the pH of the skin is often preferred, such as a pH of around 6.0-6.8, or around 6.5, such as 6.5±0.20, 6.25±0.25, or 6.0±0.25. In some embodiments, a CBD-comprising composition can be formulated with a pH of 5-7, 5-6, 5.5-6.5, or around 6. In some embodiment, the pH is around 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. In some embodiments, the pH can be around 5.2-5.8, 5.6-5.7 or around 5.5. In some embodiments, the pH is around 6-6.5. In some embodiments, the pH is greater than 6.8 or 7.0. In some embodiments, the pH is lower than 5.0.

Provision of a defined pH can be achieved using methods known in the field, comprising addition of one or more acid(s), base(s), salt(s) of said acid(s) and/or base(s), buffering agent(s) and/or pH-stabilizer(s), including any combination thereof. In some embodiments, citric acid, in particular citric acid monohydrate is used in this context. In some embodiments, other pharmaceutically acceptable acid(s) or base(s) including their salts can be used. In some embodiments, triethanolamine and/or citrate/citric acid are used in the provision and/or maintenance of the desired pH. In some embodiments, an alkanolamine such as aminomethyl propanol (AMP) is used as a buffering agent.

A “chelating agent” or “chelator” is a compound that is capable of forming chelated complexes with ions, such as metal ions. It is usually an organic compound, capable of reacting with a metal ion to produce a chelate. Examples of suitable chelating agents may comprise EDTA, citric acid, tartaric acid, phytic acid, triethanolamine, and salicylaldehyde. In some embodiments, phytic acid and/or its salt is used as chelating agent. In some embodiments, the chelator is present in a concentration of 0.075-0.2% (w/w), or around 0.1% (w/w). In some embodiments, the chelator is phytic acid or phytate, such as sodium phytate.

Generally, the presence of oil(s) and/or fat(s) is not desired in CBD-comprising compositions according to the invention, in particular in the context of a topical composition formulated as a gel to be applied onto the skin of a subject. In some embodiments, the composition is not formulated as an oil-in-water emulsion or water-in-oil emulsion. Thus, in some embodiments, the composition comprises no, or insignificant amounts, e.g. than 1.0, 0.5 or 0.1% (w/w) oil, such as edible oil, dehydrated oil, and/or dehydrated edible oil. This may seem counter intuitive, as oils/fats are commonly used to keep the skin soft and smooth, in particular in conventional wound treatment recipes, such as for skin burns, where e.g. goat fat is used for treatment burn wounds. However, in the present invention it is believed the potential downs side of a wound treatment formulation without fat(s)/oil(s) are more than outweighed by the current CBD-comprising recipes, in particular when comprising e.g. hyaluronic acid/hyaluronate. Without wanting to be bound by any theory, it is believed that the presence of such fat(s) and/or oil(s) contributes negatively with respect to the efficacy of the formulation, as CBD is hydrophobic, and oil(s)/fat(s) will form a kind of barrier and/or layer on the skin, thereby impeding relevant active compounds from being able to actively participating in the wound healing process. Furthermore, oil(s) and/or fat(s) may hinder, obstruct, or even destroy the Carbomer hydrogel, whereby the composition will no longer be able to form a protective layer covering the wound and providing a satisfactory environment for wound healing.

Consequently, in some embodiments, a CBD-comprising composition as disclosed herein comprises no or only minute amounts of oil(s) and/or fat(s). In some embodiments, the composition comprises less than 1.0, 0.5, 0.1% oil(s) and/or fat(s). In some embodiments, the composition does not comprise one or more of: (i) oil, such as edible oil, (ii) fat, such as edible fat. Generally, compositions disclosed herein do not comprise an oil-in-water or water-in-oil emulsion.

In some embodiments, a wound treatment composition, such as a wound- and/or sore composition, is formulated as a gel for topical application, comprising 0.1-5%, 0.2-2%, 0.3-1%, 0.4-0.75%, or around 0.5% (w/w) CBD; 25-85%, 35-75%, 45-70% or 55-65% (w/w) water; and one or more penetrator(s) and/or penetration enhancer(s) such as propylene glycol and/or pentylene glycol; (i) one or more gelator(s), such as polyacrylic acid/polyacrylate (e.g. 2-propeonic acid homopolymer and/or Carbomer); and optionally (ii) one or more skin moisturizers and/or skin conditioners, such as panthenol and/or allantoin; (iii) one or more further wound healing compound(s), such as hyaluronic acid and/or its salt; (iv) one or more anti-microbial agent(s), such as benzalkonium chloride; (v) one or more pH stabilizer(s) and/or buffering agent(s); such as aminomethyl propanol (AMP) and/or (vi) one or more chelating agent(s), such as phytic acid and/or its salt; including any combination(s) of (ii)-(vi).

In some embodiments, a composition as disclosed herein, such as a wound and/or sore treatment composition optionally formulated as a gel comprises component (i) and (ii), and optionally one or more of components (iii)-(vi). In some embodiments, the composition comprises component (i) and (iii), and optionally one or more of components (ii), (iv)-(vi). In some embodiments, the composition comprises component (i) and (iv), and optionally one or more of components (ii), (iii), (v), (vi). In some embodiments, the composition comprises component (i) and (v), and optionally one or more of components (ii)-(iv), and (vi). In some embodiments, the composition comprises component (i) and (vi), and optionally one or more of components (ii)-(v). Components (i)-(vi) are disclosed in detail herein.

In some embodiments, the composition comprises some alcohol. In some embodiments, the composition comprises no alcohol, or only small amounts of alcohol. In some embodiments, the composition comprises by weight 10% or less, 5% or less, 2% or less, 1% or less, 0.5% or less, 0.25% or less, or 0.1% or less alcohol(s). Usually the alcohol is a low molecular weight alcohol, such as one or more low molecular weight alcohol, such as one or more C1-C4 alcohol, such a one or more of methanol, ethanol, propanol, butanol, including any isomers and/or any combination thereof. In some embodiments, the wound treatment composition comprises no and/or 0.25% (w/w) or less, such as 0.20% (w/w) or less, or 0.10 (w/w) or less alcohol. In some embodiments, the composition comprises no C1-C4 alcohol. In some embodiments, the composition comprises 0.25% (w/w) or less, such as 0.20% (w/w) or less, or 0.10 (w/w) or less C1-C4 alcohol. Generally, in the context of the present invention, the presence of alcohol, in particular low-molecular weight alcohols, such as C1-C4 alcohols is not desired. Commonly, alcohol is used to clean and/or disinfect a wound. However, alcohol may actually harm sensitive tissue and delay healing. Furthermore, applying alcohol on a wound, in particular an open wound, and or sensitive tissue will give an unpleasant feeling of stinging and/or burning. Alcohol has also a dehydrating effect on the skin, which is undesirable.

CBD compositions, depending on the production method used, including the variety of Cannabis used. When extracted, they may comprise varying amount of impurities, such as further cannabinoids. Generally, the presence of such impurities is not desired, especially, when the nature, concentration and/or composition of these impurities is unknown, and/or when they vary significantly from batch to batch. Thus, in some embodiments, the CBD has a purity of at least 95% (w/w), 98% (w/w), 99% (w/w), 99.5 (w/w), or more than 99.8% (w/w).

A composition, such as a wound and/or sore treatment composition may comprise one or more further cannabinoid(s), such as one or more psychoactive or one or more non-psychoactive cannabinoid(s), e.g. one or r more of THC (tetrahydrocannabinol), THCA (tetrahydrocannabinolic acid), CBDA (cannabidiolic acid), CBG (cannabigerol), CBC (cannabichromene), CBL (cannabicyclol), CBV (cannabivarin), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), and or CBT (cannabicitran), including any combination(s) thereof.

However, as outlined above, significant amounts, in particular physiologically active amounts of one or more further cannabinoids is generally undesirable. Consequently, in some embodiments, the composition does not comprise, or comprises less than 1.5, 1.0, 0.5 or 0.1% (w/w) of one or more further cannabinoid(s), such as one or more psychoactive or one or more non-psychoactive cannabinoid(s), e.g. one or more of THC (tetrahydrocannabinol), THCA (tetrahydrocannabinolic acid), CBDA (cannabidiolic acid), CBN (cannabinol), CBG (cannabigerol), CBC (cannabichromene), CBL (cannabicyclol), CBV (cannabivarin), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), and or CBT (cannabicitran), including any combination(s) thereof. In some embodiments, the CBD or composition comprises less than 0.1 (w/w) THC. In some embodiments, the CBD or composition comprises less than 1.5% (w/w) of any one of CBDV, CBDA, CBG, CBN. In some embodiments, the CBD comprises less than 1.0, 0.5, 0.2 or 0.1% (w/w) by weight CBDV, CBDA, CBG, CBN, or THC.

The absence of significant amounts of any further cannabinoids appears counter intuitive and contrary to common belief claiming a positive, synergistic effect of further cannabinoids in compositions for wound- and/or pain treatment.

In some embodiments, a composition is provided, comprising:
a) 20-85% (w/w), 40-75% (w/w), 50-70% (w/w), 55-65% (w/w), around 61% (w/w) water
b) 0.1-60% (w/w), 5-55% (w/w), 10-50% (w/w), 30-50% (w/w), 35-45% (w/w), or around 30% (w/w) propylene glycol;
c) 0.1-15% (w/w), 0.5-12% (w/w), 1.0-10% (w/w), 1.5-7.5% (w/w), 2.0-6.0% (w/w), 2.5-4.0% (w/w), or around 5% (w/w) pentylene glycol;
d) 0.1-10% (w/w), 0.2-5% (w/w), 0.4-2.0% (w/w), 0.6-1.0% (w/w), or around 0.8% (w/w) polyacrylic acid and/or polyacrylate, such as sodium polyacrylate, Carbomer, and/or 2-propenoic acid homopolymer.
e) 0.1-5% (w/w), 0.15-2% (w/w), 0.2-1% (w/w), 0.25-0.75% (w/w), or around 0.5% (w/w) CBD; and optionally, CBD with a purity of at least 98% (w/w);
f) 0.1-5% (w/w), 0.15-3.0% (w/w), 0.2-1.5% (w/w), 0.3-0.8% (w/w), or around 0.5% (w/w) panthenol;
g) 0.1-5% (w/w), 0.2-2% (w/w), 0.3-1% (w/w), 0.4-0.75% (w/w), or around 0.5% (w/w) hyaluronic acid and/or hyaluronate, such as sodium hyaluronate;
h) 0.01-2.5% (w/w), 0.025-0.1.0% (w/w), 0.05-0.5% (w/w), 0.075-0.2% (w/w), or around 0.1% (w/w) benzalkonium chloride;
i) 0.01-5% (w/w), 0.05-2% (w/w), 0.1-1% (w/w), 0.2-0.5% (w/w), or around 0.3% (w/w) allantoin; and
j) 0.01-2.5% (w/w), 0.025-0.1.0% (w/w), 0.05-0.5% (w/w), 0.075-0.2% (w/w), or around 0.1% (w/w) phytic acid or phytate, such as sodium phytate.

In some embodiments, a composition is provided, comprising one or more of:
a) 50-70% (w/w), 55-65% (w/w), around 61% (w/w) water
b) 30-50% (w/w), 35-45% (w/w), or around 30% (w/w) propylene glycol;
c) 2.0-6.0% (w/w), 2.5-4.0% (w/w), or around 5% (w/w) pentylene glycol;
d) 0.4-2.0% (w/w), 0.6-1.0% (w/w), or around 0.8% (w/w) 2-Propenoic acid, homopolymer, Carbomer, polyacrylic acid and/or polyacrylate, such as sodium polyacrylate;
e) 0.2-1% (w/w), 0.25-0.75% (w/w), or around 0.5% (w/w) CBD;
f) 0.2-1.5% (w/w), 0.3-0.8% (w/w), or around 0.5% (w/w) panthenol;
g) 0.3-1% (w/w), 0.4-0.75% (w/w), or around 0.5% (w/w) hyaluronic acid and/or hyaluronate, such as sodium hyaluronate;
h) 0.05-0.5% (w/w), 0.075-0.2% (w/w), or around 0.1% (w/w) benzalkonium chloride;
i) 0.1-1% (w/w), 0.2-0.5% (w/w), or around 0.3% (w/w) allantoin; and
j) 0.05-0.5% (w/w), 0.075-0.2% (w/w), or around 0.1% (w/w) phytic acid or phytate, such as sodium phytate;
   including any combination(s) thereof.

In some embodiments, said composition comprises at least 4, 5, 6, 7, 8, 9 or all 10 of constituents (a)-(j). In some embodiments, said composition comprises components/constituents (a)-(c), and at least 1, 2, 3, 4, 5, 6 or all 7 of components (d)-(j). In some embodiments, said formulation is formulated as a gel and comprises components (a)-(d), and optionally at least 1, 2, 3, 4, or all 5 of components (e)-(j). Such composition can e.g. be formulated with a near neutral, usually slightly acidic pH, such as around 6.0-6.9, 6.2-6.8, 6.4-6.6, or around 6.5.

In some embodiments, a composition is provided, comprising:
a) 55-65% (w/w), around 61% (w/w) water
b) 35-45% (w/w), or around 30% (w/w) propylene glycol;
c) 2.5-4.0% (w/w), or around 5% (w/w) pentylene glycol;
d) 0.6-1.00% (w/w), or around 0.8% (w/w) polyacrylic acid and/or polyacrylate;
e) 0.25-0.75% (w/w), or around 0.5% (w/w) CBD;
f) 0.3-0.8% (w/w), or around 0.65% (w/w) panthenol;
g) 0.4-0.75% (w/w), or around 0.5% (w/w) hyaluronic acid and/or hyaluronate, such as sodium hyaluronate;
h) 0.075-0.2% (w/w), or around 0.1% (w/w) benzalkonium chloride;
i) 0.2-0.5% (w/w), or around 0.3% (w/w) allantoin; and
j) 0.075-0.2% (w/w), or around 0.1 (w/w) phytic acid or phytate, such as sodium phytate.

In some embodiments, said formulation is formulated as a gel, and/or possesses a near neutral, usually slightly acidic pH, such as around 6.0-6.9, 6.2-6.8, 6.4-6.6, or around 6.5.

In some embodiments the CBD used in the provision of the composition, such as a topical wound healing composition, is crystalline, such as “type A CBD” as disclosed herein. In some embodiments, said CBD is provided as—or capable of forming—needle-like crystals.

In some embodiments, a CBD-comprising composition according to the first aspect may comprise a further cannabinoid, such as a one or more cannabinoid(s) selected from: THC (tetrahydrocannabinol), THCA (tetrahydrocannabinolic acid), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), including one or more cannabinoids of the following types: CBG-type, CBC-type, “CBD-type other than CBD”. THC-type, CBN-type, CBE-type, iso-THC-type, CBL-type, CBT-type, including any combination(s) thereof. Such further cannabinoid may comprise hallucinogenic and/or non-hallucinogenic cannabinoids. Generally, non-hallucinogenic cannabinoids are preferred in order to avoid undesired side-effects upon use or treatment with composition(s) comprising such compounds, in particular when they are present in physiologically active amounts.

Further suitable concentrations and/or concentration ranges may be disclosed herein.

Concerning the CBD used in the preparation or formulation of a CBD-comprising composition, such as a topical formulation, in some embodiments, the CBD used in the provision of the composition is crystalline.

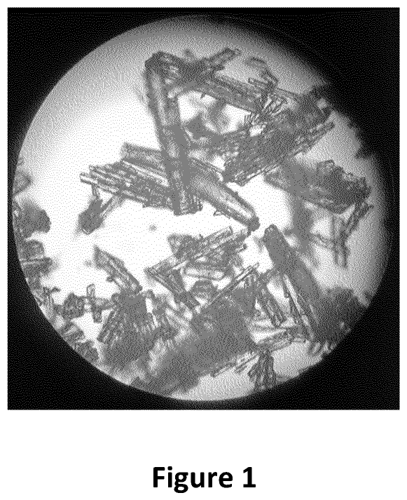

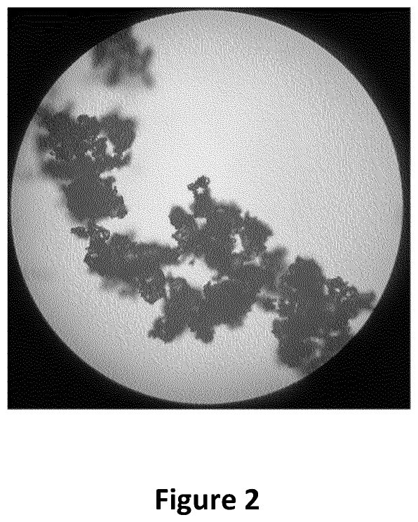

In some embodiments, the CBD used for providing a composition as disclosed above is characterized by a certain features, such as the crystal structure and/or conformation. It has been observed by the inventors, see e.g. Example 12, that CBD with a needle-like crystal structure (=crystal structure A; see FIG. 1), surprisingly and unexpectedly, appears significantly more potent than CBD with a different crystal structure, a non-needle like structure, also termed “bunch-like or “cluster-like” herein (=crystal structure B; see FIG. 2).

In some embodiments, the CBD possesses, when crystalline, or is capable of forming a needle-like crystal structure. In some embodiments, CBD of crystal structure A (or capable of forming needle-like crystals) is at least 1.2, 1.5, 2, 3, 4, 5, 7.5, 10, 15 or 20 times more “potent” on a weight/weight basis than CBD of crystal structure B (or capable of forming cluster/bunch-like crystals). Often, the “A-type” CBD is at least 2.5, 5, 7.5, or 10 times more “potent” on a weight/weight basis than “B-type” CBD. The “potency” of “type A” CBD can e.g. be determined in measuring the time needed for wound and/or soar healing using essentially identical compositions with the exception of the type of CBD used. Alternatively, the potency can be determined in the quantity of CBD needed to provide the same effect, such as wound and/or sore healing time. In some embodiments, the use of a more potent CBD results in a reduced wound and/or soar healing time. In some embodiments, the use of a more potent CBD allows for a reduction of the quantity of CBD used in the formulation of the wound and/or soar healing composition. In some embodiments, the use of a more potent CBD allows for a reduction of the quantity of formulation necessary to provide a comparable wound and/or soar healing effect.

CBD of crystal structure A, or CBD capable of forming needle-like crystals, is also called “type A CBD” herein, while CBD of crystal structure B, or CBD capable of forming “bunch-like or “cluster-like” crystals is called “type B CBD”. In some embodiments, the CBD is “type A CBD”. Often, “type A CBD” is preferred in contrast to “type B CBD”.

It can be speculated, if the CBD needs to be in an active form, such one or more specific conformation(s) in order to be active upon administration to a subject, such as in a topical formulation. Lack of activity or potency can also be caused by a lower uptake rate and/or difficulties in passing through the skin.

Without wanting to be bound by any theory, it is believed that the difference in crystal structure may be caused by a different molecular structure, such as a different conformation. This could e.g. be due to a failure of the subject's body to recognize the “wrong” CBD conformation or the like. It is conceivable that the differences in CBD crystal structure are caused by a different extraction process. In particular, the CBD disclosed in FIG. 1 was provided by an extraction process, comprising extraction with isopropanol, distillation and crystallization with heptane (see e.g. Example 12), while the CBD disclosed in FIG. 2 was provided by critical CO₂ extraction.

Generally, crystalline CBD can be provided by methods and techniques known in the art, such as by methods disclosed in U.S. Ser. No. 10/413,845 and/or U.S. Ser. No. 10/414,709.

In short, crystalline CBD can be provided from hemp or cannabis (Cannabis sativa) by a method consisting essentially of:
Extracting hemp or cannabis with e.g. isopropanol to produce an extract rich in cannabinoids, THC, CBD and terpenes
Evaporating the solvent portion of the extract to generate a substantially solvent-free extract
Distilling the substantially solvent-free extract to isolate the CBD, and
Crystallizing the distilled, isolated CBD to produce a crystallized, isolated CBD and one or more recrystallization(s) if needed by the use of a suitable organic solvent, such as an alkane, e.g. heptane, commonly followed by
Solvent removal by e.g. vacuum drying, to remove volatile remnants.

Thus, in some embodiments, the CBD crystals used in the formulation of the topical composition are needle-like crystals, such as crystals shown in FIG. 1. Likewise, in some embodiments, the CBD crystals used in the formulation of the topical composition are not cluster- or bunch-shaped, such as crystals similar to crystals shown in FIG. 2.

In some embodiments, the CBD crystals used in the formulation of the topical composition are not provided by an extraction method comprising critical CO₂ extraction.

In some embodiments, the CBD crystals used in the formulation of the topical composition are provided by a method comprising extraction with a C₃-C₄ alcohol, such as isopropanol, and one or more crystallisations steps with a C₆-C₈ alkane, such as heptane. In some embodiments, the C₃-C₄ alcohol is isopropanol. In some embodiments, the C₆-C₈ alkane is heptane. In some embodiments, the C₃-C₄ alcohol is isopropanol, and the C₆-C₈ alkane is heptane. This combination is believed to provide CBD crystals of satisfactory quality, such as absence or reduction in inhibitors and/or the desired conformation of the CBD.

In some embodiments, a suitable CBD product can be obtained when the CBD crystals are provided by a method comprising critical CO₂ extraction and one or more crystallisations steps with a C₆-C₈ alkane, such as heptane.

As seen in Table 1, it can be seen that the Cannabinoid profile of type A and type B CBD can be rather similar.

TABLE 1
Analysis of CBD of crystal structure A versus crystal structure B

| Cannabinoid | | |
|---|---|---|
| profile | Type A | Type B |
| CBD | 99.33% | 98.60% |
| CBDV | 0.39% | 0.19% |
| CBDA | 0.01% | n.d. |
| CBG | n.d. | n.d. |
| CBN | 0.04% | n.d. |
| THC | n.d. | n.d. |

n.d. not detected;
type A CBD was sourced from Enecta,
type B CBD was sourced from Pharma Hemp

It is, however, also conceivable that the differences in crystal structure, can relate to and be caused by different extraction processes. Different crystal structures can also be indicative of different concentrations of “CBD inhibitors”, and/or different concentrations of “CBD enhancers”. In some embodiments, terpenes, such as naturally occurring terpenes, in particular terpenes found in plants, such as in Cannabis sativa, act as CBD inhibitors, which is not desirable.

Thus, in some embodiments, CBD of crystal structure B alias “type B CBD” can be converted to CBD of crystal structure A alias “type A CBD” (and/or CBD capable of forming crystal structure A) by an organic extraction step and/or recrystallisation step. In such embodiments, it is conceivable that the change in crystal structure is related to the presence of inhibitors that are reduced significantly in the additional extraction and/or crystallization step(s). Alternatively, the organic extraction step may provide a change in conformation of the CBD, rendering it more active again. In some embodiments, recrystallization with heptane can change the B-type CBD into A-type CBD.

In some embodiments, CBD of crystal structure B has been provided by critical CO₂ extraction, such as CBD crystals provided by www.pharma-hemp.com and/or following a similar extraction protocol as said manufacturer.

In some embodiments, presence of terpenes and/or terpenoids, in particular Cannabis sativa terpenes or in a CBD-comprising topical composition as disclosed herein, provides one or more undesirable effect(s), such as one or more of: reduced efficiency or potency, inability or reduced ability to recognize the CBD, need for a higher CBD formulation for obtaining similar effect, increase in non-CBD cannabinoids in the formulation. In some embodiments, said composition comprises 0.0001% or less, 0.001% or less, 0.01% or less, or 0.1% or less terpenes, in particular Cannabis sativa terpenes, by weight.

In some embodiments, the crystalline CBD does not comprise significant amounts of terpenes, such as less than 0.1, less than 0.05, less than 0.02, less than 0.01, less than 0.005, less than 0.002, less than 0.001% terpenes by weight.

It is also conceivable that other plant components, such as terpenoids can act as inhibitors. In some embodiments the presence of terpenoids, such as Cannabis sativa terpenoids can be undesirable. In some embodiments, the crystalline CBD does not comprise significant amounts of terpenoids, such as less than 0.1, less than 0.05, less than 0.02, less than 0.01, less than 0.005, less than 0.002, less than 0.001% terpenoids by weight.

In some embodiments, the use of CBD having or capable of providing crystals of crystal structure A, such as shown in FIG. 1 in a CBD-comprising composition as disclosed herein, provides a positive effect, such as one or more of: increased efficiency, possibility to reduce total amount of CBD in the formulation, the subject needs less topical composition, such as wound and/or soar healing formulation to achieve the same effect, improved recognition and/or CBD uptake by the subject's body, reduction in non-CBD cannabinoids in the formulation and/or other impurities.

Generally, compositions according to the first aspect can be provided using methods, procedures and/or unit operations known in the art. In some embodiments, compositions according to the first aspect can be provided as shown in the second aspect.

In a second aspect, the present invention relates to a method for providing a composition according to the first aspect, such as a topical wound treatment composition formulated as a gel, comprising the steps or acts of:
i. providing CBD preferably in solid form, such as crystalline and/or powder form and/or with a purity of at least 95% (w/w), 98% (w/w), 99% (w/w), 99.5% (w/w), or more than 99.8% (w/w);
ii. dissolving the CBD from step (a) in a penetrator and/or penetration enhancer, such as propylene glycol;
iii. providing an aqueous gel, such as by dissolving hyaluronic acid and/or its salt in water;
iv. combining the dissolved CBD from step (b) with the aqueous gel from step (c); and
v. adding and mixing the remaining ingredients to provide the composition; optionally
vi. adjusting pH to a desired set value, such as between 6.0 and 6.8, such as around 6.5; and/or
vii. aliquoting the composition into receptacles.

In some embodiments, the remaining ingredients(s) is/are one or more of:
a) 20-85% (w/w), 40-75% (w/w), 50-70% (w/w), 55-65% (w/w), around 61% (w/w) water;
b) 0.1-60% (w/w), 5-55% (w/w), 10-50% (w/w), 30-50% (w/w), 35-45% (w/w), or around 30% (w/w) propylene glycol;
c) 0.1-15% (w/w), 0.5-12% (w/w), 1.0-10% (w/w), 1.5-7.5% (w/w), 2.0-6.0% (w/w), 2.5-4.0% (w/w), or around 5% (w/w) pentylene glycol;
d) 0.1-10% (w/w), 0.2-5% (w/w), 0.4-2.0% (w/w), 0.6-1.0% (w/w), or around 0.8% (w/w) Carbomer, polyacrylic acid homopolymer and/or polyacrylate, such as sodium polyacrylate;
e) 0.1-5% (w/w), 0.15-2% (w/w), 0.2-1% (w/w), 0.25-0.75% (w/w), or around 0.5% (w/w) CBD;
f) 0.1-5% (w/w), 0.15-3.0% (w/w), 0.2-1.5% (w/w), 0.3-0.8% (w/w), or around 0.5% (w/w) panthenol;
g) 0.1-5% (w/w), 0.2-2% (w/w), 0.3-1% (w/w), 0.4-0.75% (w/w), or around 0.5% (w/w) hyaluronic acid and/or hyaluronate, such as sodium hyaluronate;
h) 0.01-2.5% (w/w), 0.025-0.1.0% (w/w), 0.05-0.5% (w/w), 0.075-0.2% (w/w), or around 0.1% (w/w) benzalkonium chloride;
i) 0.01-5% (w/w), 0.05-2% (w/w), 0.1-1% (w/w), 0.2-0.5% (w/w), or around 0.3% (w/w) allantoin; and
j) 0.01-2.5% (w/w), 0.025-0.1.0% (w/w), 0.05-0.5% (w/w), 0.075-0.2% (w/w), or around 0.1% (w/w) phytic acid or phytate, such as sodium phytate;
   and optionally one or more pharmaceutically acceptable adjuvant(s).

In some embodiments, the CBD is crystalline CBD. In some embodiments, the CBD is “type A CBD”. Often, the use of “type A CBD” is preferred in contrast to “type B CBD” or other types of CBD.

In a third aspect, the present invention pertains to a composition, such as a wound treatment composition provided according to the second aspect.

In a fourth aspect, the present invention concerns a receptacle comprising a composition, such as a wound treatment composition according to the first, third or seventh aspect.

It can be desirable to protect a composition as disclosed herein, such as a wound treatment composition e.g. formulated as a gel, from damaging influence of visible and/or UV-light. Thus, in some embodiments, the receptacle is adapted to provide light and/or UV protection to the wound treatment composition.

In a fifth aspect, the present invention relates to a kit comprising a receptacle according to the fourth aspect, and optionally, an instruction for use.

In some embodiments, the kit comprises a packaging, such as carton or the like for said receptacle and/or instruction for use. In some embodiments, said packaging may provide light and/or UV protection, such as additional protection during storage.

In a sixth aspect, the present invention pertains to a method for treatment of a wound or sore of a subject, comprising topical application of a composition according to the first, third or seventh aspect.

In some embodiments, the subject is an animal or a human. In some embodiments, the subject is a human, such as a female, male, senior, adult, adolescent, child, or infant. In some embodiments, the subject is an animal, such as one or more of pet, husbandry, mammal, reptile, bird, and/or animal in a zoo. In some embodiments, the subject is a mammal.

Generally, treatment comprises topical application of a suitable amount of the wound/sore treatment composition. In some embodiments, the dosage regimen and/or suitable amount is around 0.25 g, such as roughly around the size of a pea, per 20-30 cm² per application. Application of a suitable amount of the composition provides a thin film or layer of said composition on the applicated area, covering the wound and usually also some surrounding area.

In some embodiments, said treatment comprises more than one application of the treatment composition. In some embodiments, treatment comprises application of the wound treatment composition once or several times per day. In some embodiments, the wound treatment composition is applied 1×, 2×, 3×, 4× or more than 4× per day. In some embodiments, the composition is applied as needed. In some embodiments, the composition is commonly applied 3-4 timers a day.

In some embodiments, the wound, sore and/or rash is selected from one or more of: clean wound, contaminated wound, infected wound, colonized wound; related to a medical treatment, cosmetical treatment, surgery, tattooing; incision wound, a laceration, an abrasion, an avulsion, a puncture wound, a gunshot wound; burn wound, such as a first-, second-, third- or fourth-degree burn; sunburn, UV light; frost bite, such as a first-, second-, third- or fourth-degree frost bite; blister; chemical wound through contact with a chemical; rash; allergy; related to a medical condition; sore, such as a bay sore, bed sore, canker sore, colds sore, desert sore, pressure sore, saddle sore, summer sore, veldt sore or venereal sore; an animal bite, animal sting, such as bite and/or sting by an invertebrate, such as an insect, e.g. mosquito, bee, wasp, hornet, fly, ant, spider, coral, jellyfish, aquatic stinging animals, poisonous animal, poisonous fish; including any combination thereof.

In some embodiments, the wound is, is related to, and/or caused by one or more of: clean wound, a contaminated wound, an infected wound, or a colonized wound. In some embodiments, the wound is, is related to, and/or caused by one or more of: medical and/or cosmetical treatment, surgery, tattooing. In some embodiments, the wound is, is related to, and/or caused by one or more of: an incision wound, a laceration, an abrasion, an avulsion, a puncture wound, a gunshot wound. In some embodiments, the wound is, is related to, and/or caused by one or more of: a burn wound, a first-, second-, third- and/or fourth-degree burn. In some embodiments, the wound is, is related to, and/or caused by sunburn, UV light, and/or electromagnetic radiation. In some embodiments, the wound is, is related to, and/or caused by one or more: frost bite, first-, second-, third- and/or fourth-degree frost bite. In some embodiments, the wound or sore is, is related to, and/or caused by a blister. In some embodiments, the wound is, is related to, and/or caused by contact with a chemical. In some embodiments, the wound is, is related to, and/or caused by a rash. In some embodiments, the wound is, is related to, and/or caused by an allergy. In some embodiments, the wound or sore is, is related to, and/or caused by a medical condition. In some embodiments, the wound is, is related to, and/or caused by one or more of: a sore, a bay sore, bed sore, canker sore, colds sore, desert sore, pressure sore, saddle sore, summer sore, veldt sore or venereal sore. In some embodiments, the wound is, is related to, and/or caused by one or more of: animal bite, animal sting, invertebrate, insect, mosquito, bee, wasp, hornet, fly, ant, or spider, coral, jellyfish, aquatic stinging animals, and/or poisonous fish.

In a seventh aspect, the present invention concerns a composition according to the first or third aspect for use as a medicament, and/or cosmetics. This may comprise treatment of a skin condition or disease, such as one or more of a wound, sore and/or rash selected from one or more of: clean wound, contaminated wound, infected wound, colonized wound; related to a medical treatment, cosmetical treatment, surgery, tattooing; incision wound, a laceration, an abrasion, an avulsion, a puncture wound, a gunshot wound; burn wound, such as a first-, second-, third- or fourth-degree burn; sunburn, UV light-related burn, electromagnetic radiation-related burn; frost bite, such as a first-, second-, third- or fourth-degree frost bite; blister; chemical wound through contact with a chemical; rash; allergy; related to a medical condition; sore, such as a bay sore, bed sore, canker sore, colds sore, desert sore, pressure sore, saddle sore, summer sore, veldt sore or venereal sore; an animal bite, animal sting, such as bite and/or sting by an invertebrate, such as an insect, e.g. mosquito, bee, wasp, hornet, fly, ant, spider, coral, jellyfish, aquatic stinging animals, poisonous animal, poisonous fish; including any combination thereof.

Compositions as disclosed herein possess surprising and unexpected properties. Test results show a surprisingly good results and appear not only to be comparable with traditional formulations, but also even faster and/or better with respect to wound and/or sore healing. In some embodiments, a topical composition according to the present invention is comparable and/or better than e.g.: (a) Hansaplast Wound Healing Ointment, comprising white petrolatum, thin paraffin oil, ceresin wax, glycerin, panthenol, glyceryl stearate; (b) Klinion L-Mesitran, comprising medical honey, water, sunflower oil, vitamin A, vitamin C, vitamin E, and purified lanolin (medilan); and/or (c) Aloe Vera gel AVIVIR, comprising Aloe barbadensis leaf extract, Xanthan gum, Sodium benzoate, Potassium sorbate, and Citric acid.

Without wanting to be bound by any theory, it is believed that a high content of a permeator, such as a polyol, glycol, in particular propylene and/or pentylene glycol contributes to the positive effects of the wound treatment compositions of the present invention. In some embodiments, the permeator provides a stability-enhancing effect to the gel, and/or an improvement with respect to wound healing, such as a reduction in time for wound healing.

Generally, wound treatment formulations, such as gels disclosed herein are efficient and comparably to, or even better than common formulations/products on the market. The formulations of the present invention provide one or more additional effect in terms of a soothing feeling when applying the gel by external moisturizing and cooling, as well as providing additional a pain relief, apart from efficient wound healing. Furthermore, the topical formulations appear to provide a reliable and beneficial effect in terms of wound environment and wound healing, reducing the risk of complications, such as infections and the like, and no or reduced scar-formation. Thus, in some embodiments, application or use of a wound composition as disclosed herein, such as a wound composition formulated as a gel, provides one or more of: faster healing; pain relief, formation of a protective layer on wound, formation of a physical barrier; biocompatibility, non-toxicity, fits and can be used to fill and/or treat irregular wound shapes, provides a moist environment, provides and/or draws humidity to the wound, provides a disinfecting effect, prevents infection, removes excess exudate, provides a good wound healing environment, reduces or eliminates scar formation, and/or is easy to use and/or apply, including any combination thereof.

In an eighth aspect, the present invention concerns a CBD-comprising composition, wherein the CBD used in the formulation is crystalline and/or of “type A”. In some embodiments, said composition is a topical composition as disclosed herein, such as a wound and/or sore healing composition according to the first, third and/or seventh aspect. In some embodiments, the CBD is of type A (needle-like crystals) or capable of forming needle-like crystals. as disclosed in e.g. the first aspect and/or in the Examples.

In a ninth aspect, the present invention pertains to a dosage regimen, comprising administering a topical composition, in particular CBD-comprising topical composition as disclosed herein. In some embodiments, the CBD is of “type A”.

The present invention is described in more detail and specifically with reference to the Examples, which however are not intended to limit the present invention.

### EXAMPLES

Percentages are % by weight. Generally, crystalline CBD is sourced from Enecta, unless indicated otherwise

### Example 1—Provision of Wound Treatment Compositions

CBD-comprising compositions according to the present invention can be provided using methods, unit operations, protocols and/or know-how customary in the field. This can e.g. be performed as disclosed herein, such as according to the third aspect of the invention, and/or in particular, according to the following Examples.

CBD data sheet

| TEST | SPECIFICATION |
|---|---|
| Identification (NMR) | Positive |
| Purity (HPLC) | ≥98.0% |
| Related substances (Purity HPLC): | |
| CBDV | ≤1.5% |
| CBDA | ≤1.5% |
| CBG | ≤1.5% |
| CBN | ≤1.5% |
| THC | ≤0.10% |
| Water content (KF) | ≤1.0% |
| Specific Rotation | −135.0 ± 5.0° |
| Melting point | 66.0 ± 3.0° C. |
| Total ash | ≤0.30% |

CBDV = Cannabidivarin (also called Cannabidivarol),
CBDA = Cannabidiolic acid,
CBG = Cannabigerol,
CBN = Cannabinol,
THC = Tetrahydrocannabinol

Step I: CBD, at least 98% (w/w) pure, such as crystalline CBD in powder form provided from Enecta® is dissolved in propylene glycol.
Step II: Hyaluronic acid sodium salt is dissolved in water under agitation to provide a gel.
Step III: The propylene glycol and the gel fraction are combined under agitation.
Step IV: The remaining components are added under agitation to provide the wound treatment composition.
Step V (optional): pH adjustment to e.g. pH 6.5 if needed.

Formulation A “complete”

| | | Product | Purity | Weight |
|---|---|---|---|---|
| Product name | INCI name | # | % (w/w) | g |
| Water demin. | Aqua | 009-00001 | 100 | 60.95 |
| Propylenglykol USP | Propylene Glycol | 009-00162 | 100 | 30 |
| Hydrolite-5 616751, | Pentylene Glycol | 009-00338 | 100 | 5 |
| Pentylene Glycol | | | | |
| 111900, Microcare | | | | |
| Emollient PTG | | | | |
| Rheocare Plus - | Carbomer | 009-00084 | 100 | 0.8 |
| 2-Propenoic acid, | | | | |
| homopolymer - | | | | |
| Highly crosslinked | | | | |
| polyacrylic acid | | | | |
| CBD | Cannabidiol | 034-00358 | 100 | 0.5 |
| Panthenol 75% USP | Panthenol | 009-00980 | 75 | 0.65 |
| Hyaluronsaure, | Sodium Hyaluronate | 009-00297 | 100 | 0.5 |
| Na-Salz 1.0-1.5 Mio. | | | | |
| Benzalkoniumchlorid | Benzalkonium | 009-00567 | 50 | 0.2 |
| Microcare QT | Chloride, Aqua | | | |
| Allantoin | Allantoin | 009-00008 | 100 | 0.3 |
| Dermofeel PA | Phytic Acid | 009-00633 | 50 | 0.2 |
| AMP Ultra | Aminomethyl Propanol | | 100 | 0.9 |
| | | | | 100 |

Formulation B “minimal”

| | | Product | Purity | Weight |
|---|---|---|---|---|
| Product name | INCI name | # | % (w/w) | g |
| Water demin. | Aqua | 009-00001 | 100 | 67.8 |
| Propylenglykol USP | Propylene Glycol | 009-00162 | 100 | 30 |
| Rheocare Plus - | Carbomer | 009-00084 | 100 | 0.8 |
| 2-Propenoic acid, | | | | |
| homopolymer - | | | | |
| Highly crosslinked | | | | |
| polyacrylic acid | | | | |
| CBD Sunlife | Cannabidiol | 034-00358 | 100 | 0.5 |
| AMP Ultra | Aminomethyl Propanol | | 100 | 0.9 |
| | | | | 100 |

Formulation C - placebo

| | | Product | Purity | Weight |
|---|---|---|---|---|
| Product name | INCI name | # | % (w/w) | g |
| Water demin. | Aqua | 009-00001 | 100 | 98.3 |
| Rheocare Plus - | Carbomer | 009-00084 | 100 | 0.8 |
| 2-Propenoic acid, | | | | |
| homopolymer - | | | | |
| Highly crosslinked | | | | |
| polyacrylic acid | | | | |
| AMP Ultra | Aminomethyl Propanol | | 100 | 0.9 |
| | | | | 100 |

### Example 2—Application/Use of Wound Treatment Compositions

The wound and/or sore treatment composition (the “wound gel”) can be applied as disclosed herein, such according to the sixth aspect of the invention.

Generally, a suitable amount, such as around 0.25 g (˜size of a pea) is applied per 20-30 cm² per application. Application of a suitable amount of the composition provides a thin film or layer of said composition on the applicated area, covering the wound and usually also some surrounding area.

The application is applied as needed, such as 3-4 times per day.

### Example 3—Test Set-Up

Inclusion criteria:
Human subjects with scraps, tattoos, burns, sunburns.
Subjects not taking medicine, except NSAID for pain.
Subjects of all ages, sex or disability.

### Clinical Outcomes

Complete wound healing
Time to complete wound healing or almost healed.

### Exclusion Criteria:

Human subjects with surgical or traumatic wounds.
Tests reporting wound healing rates without also reporting complete wound healing were excluded.

### Example 4—Test Results

TABLE A
Formulation A

| Testperson | 1 | 2 | 3 | 4 | 5 | 6 |
|---|---|---|---|---|---|---|
| Wound spot size | Deep scrape | Minor burn | Deep scrape | Tattoo on | Mild sunburn | Small Scrape |
| and position | on knee | right hand | on elbow | the back | forehead | on the top of |
| | 5*6 cm | 2*3 cm | 3*3 cm | −10*6 cm | 5*8 cm | head 1*2 cm |
| Test period(days) | 7 | 8 | 6 | 9 | 2 | 3 |
| minimum 2 days- | | | | | | |
| max 30 days | | | | | | |
| Pain or Ich scaled | B: 8 | B: 7 | B: 8 | B: 7 | B: 8 | B: 6 |
| on a 0-10 scale | A: 3 | A: 2 | A: 3 | A: 3 | A: 2 | A: 1 |
| before and after | | | | | | |
| first application | | | | | | |
| Wound spot size in | Healed | Healed | Healed | Healed | Healed | Healed |
| cm after test period | | | | | | |
| Medication reduced | NA | NA | NA | NA | NA | NA |
| during test period? | | | | | | |

TABLE B
Formulation B

| Testperson | 1 | 2 | 3 | 4 | 5 | 6 |
|---|---|---|---|---|---|---|
| Wound spot size | Tattoo on | Deep | Small scrape | Mild burn | Deep Scrape | Tattoo on |
| and position | right foot | scrape knee | on elbow | on left hand | on neck | left shoulder |
| | 2*2 cm | 4*5 cm | 3*2 cm | 1*2 cm | 2*5 cm | 6*7 cm |
| Test period(days) | 11 | 8 | 2 | 3 | 10 | 13 |
| minimum 2 days- | | | | | | |
| max 30 days | | | | | | |
| Pain or Ich scaled | B: 8 | B: 8 | B: 6 | B: 8 | B: 9 | B: 8 |
| on a 0-10 scale | A: 5 | A: 6 | A: 3 | A: 5 | A: 6 | A: 4 |
| before and after | | | | | | |
| first application | | | | | | |
| Wound spot size in | Healed | Healed | Healed | Healed | Healed | Healed |
| cm after test period | | | | | | |
| Medication reduced | NA | NA | NA | NA | NA | NA |
| during test period? | | | | | | |

TABLE C
Formulation C

| Testperson | 1 | 2 | 3 | 4 | 5 | 6 |
|---|---|---|---|---|---|---|
| Wound spot/type | Minor burn | Small Scrape on | Deep Scrape | Deep Scrape | Tattoo upper | Small Scrape |
| size and | on hand | top of head | on Elbow | on knee | right arm | on right hand |
| position | 3*3 cm | 2*2 cm | 5*5 cm | 4*4 cm | 6*5 cm | 3*2 cm |
| Test period(days) | 14 | 7 | 14 | 14 | 21 | 7 |
| minimum 2 days- | | | | | | |
| max 30 days | | | | | | |
| Pain or Ich scaled | B: 7 | B: 8 | B: 8 | B: 9 | B: 9 | B: 6 |
| on a 0-10 scale | A: 6 | A: 6 | A: 7 | A: 7 | A: 8 | A: 5 |
| before and after | | | | | | |
| first application | | | | | | |
| Wound spot size in | Healed | Healed | Smaller | Healed | Smaller but | Healed |
| cm after test period | | | slightly | | inflamed | |
| | | | inflamed | | | |
| Medication reduced | NA | NA | NA | NA | NA | NA |
| during test period? | | | | | | |

### Example 5—Provision of CBD by Alcohol Extraction, Distillation and Crystallization

Crystalline CBD can be provided by methods and techniques known in the art, such as by methods disclosed in U.S. Ser. No. 10/413,845 and/or U.S. Ser. No. 10/414,709.

In short, crystalline CBD can be provided from hemp or cannabis (Cannabis sativa) by a method consisting essentially of:
Extracting hemp or cannabis with a solvent selected from the group consisting of propanol, isopropanol, butanol, pentanol, hexanol, heptanol, and octanol to produce an extract consisting essentially of an extracted hemp or cannabis consisting essentially of tetrahydrocannabinol, a terpene, or cannabidiol;
Evaporating the solvent portion of the extract to generate a substantially solvent-free extract comprising CBD;
Distilling the substantially solvent-free extract to isolate the CBD, and
Crystallizing the distilled, isolated CBD to produce a crystallized, isolated CBD.

Often, the crystallized, isolated CBD is subjected to vacuum drying to remove volatile remnants, in particular the solvent used in crystallizing or re-crystallizing, if needed.

In particular, a method comprising extraction with isopropanol and crystallization by the use of heptane, including one or more optional re-crystallization steps, followed by vacuum drying can provide CBD with crystal structure A, i.e. needle like crystals. Furthermore, such a CBD can be very low in undesired compounds, such as terpenes.

GC chromatography or other analytical methods known in the art can be used to monitor the process such as to ensure a high yield and/or a high purity of the desired product.

Concerning the raw material, hemp comprising e.g. 2-3% CBD is dried and ground before extraction with isopropanol, such as food grade isopropanol.

Guidance for choosing the appropriate reaction based on the boiling points or ranges of the different compounds can e.g. be found here: www.nwsci.com/customer/docs/SKUDocs/RMR/Technical%20Data_Extractions_03.28.18.pdf.

CBD with crystal structure A can e.g. be provided from www.enecta.com, and/or following a similar extraction and/or purification protocol as said manufacturer.

### Example 6—Comparison of Compositions Formulated with Different Crystalline CBDs

Two compositions are prepared according to Example 1, e.g. formulation A or B, the only difference being that the crystalline CBD used in the formulation is either of type A (needle-like crystals; FIG. 1) or type B (bunch/cluster-like; FIG. 2).

Type A crystalline CBD is sourced from Enecta, while type B CBD is sourced from Pharma Hemp.

When testing both compositions, surprisingly and unexpectedly, it is seen and/or it can be concluded that type A CBD is significantly more active than type B CBD.

1.-57. (canceled)

58. A composition formulated as a wound treatment composition for topical application comprising by weight:
a) 0.1-5%, 0.2-2%, 0.3-1%, 0.4-0.75%, or around 0.5% (w/w) cannabidiol (CBD);
b) 25-85%, 35-75%, 45-70% or 55-65% (w/w) water; and
c) one or more penetrator(s).

59. Composition according to claim 58, wherein the CBD used in the provision of the CBD-comprising composition has a purity of at least 95% (w/w), 98% (w/w), 99% (w/w), 99.5 (w/w), or more than 99.8% (w/w) and is crystalline; and wherein the CBD crystals are selected from one or more of:
needle-like crystals;
crystals not provided by an extraction method comprising critical CO₂ extraction; and
crystals provided by a method comprising extraction with a C₃-C₄ alcohol, and one or more crystallisations steps with a C₆-C₈ alcohol; or
any combination thereof.

60. Composition according to claim 58, further comprising one or more components selected from:
i. One or more gelator(s);
ii. One or more skin moisturizer(s), skin conditioner(s), or skin moisturizer(s) and skin conditioner(s);
iii. One or more further wound healing compound(s);
iv. One or more anti-microbial agent(s);
v. One or more pH stabilizer(s); and
vi. One or more chelating agent(s); or
vii. any combination(s) of (i)-(vi).

61. Composition according to claim 60, wherein the composition comprises 10% (w/w) or less, 5% (w/w) or less, 2% (w/w) or less, 1% (w/w) or less, or 0.5% (w/w) or less, 0.25% (w/w) or less, or 0.1% or less C₁-C₄ alcohol selected from one or more of: methanol, ethanol, propanol, butanol, including any isomers and combinations thereof.

62. Composition according to claim 60, wherein the composition is formulated as a gel comprising one or more gelator(s) selected from: polyacrylic acid/polyacrylate, 2-propeonic acid homopolymer, and Carbomer, including any combination thereof, wherein the one or more gelator(s) is provided in a concentration of 0.1-5%, 0.2-3% (w/w), 0.5-2% (w/w), 0.6-1.0% (w/w), or around 0.8% (w/w).

63. Composition according to claim 60, wherein the composition has a pH of 6.5±0.25, 6.25±0.25, or 6.0±0.25.

64. Composition according to claim 58, wherein the composition is not formulated as an oil-in-water emulsion or water-in-oil emulsion.

65. Composition according to claim 58, wherein the composition comprises less than 1.0, 0.5 or 0.1% (w/w) oil.

66. Composition according to claim 58, wherein the composition comprises one or more further cannabinoid(s) selected from one or more of: THC (tetrahydrocannabinol), THCA (tetrahydrocannabinolic acid), CBDA (cannabidiolic acid), CBN (cannabinol), CBG (cannabigerol), CBC (cannabichromene), CBL (cannabicyclol), CBV (cannabivarin), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), and CBT (cannabicitran), including any combination(s) thereof.

67. Composition according to claim 59, wherein the composition comprises less than 1.5, 1.0, 0.5 or 0.1% (w/w) of one or more further cannabinoid(s) selected from one or more of: THC, THCA, CBDA, CBN, CBG, CBC, CBL, CBV, THCV, THCP, CBDV, CBCV, CBGV, CBGM, CBE, CBT, or any combination thereof.

68. Composition according to claim 60, wherein the one or more further wound healing compound(s) is or comprises hyaluronic acid or its salt; and wherein the one or more further wound healing compound(s) is provided in a concentration of 0.1-5% (w/w), 0.2-2% (w/w), 0.3-1% (w/w), 0.4-0.75% (w/w), or around 0.5% (w/w).

69. Composition according to claim 60, wherein the one or more penetrator(s) is or comprises propylene glycol, pentylene glycol, or both propylene glycol and pentylene glycol; said propylene glycol being provided in a concentration of 0.1-60% (w/w), 5-55% (w/w), 10-50% (w/w), 30-50% (w/w), 35-45% (w/w), or around 30% (w/w); and said pentylene glycol being provided in a concentration of 0.1-15% (w/w), 0.5-12% (w/w), 1.0-10% (w/w), 1.5-7.5% (w/w), 2.0-6.0% (w/w), 2.5-4.0% (w/w), or around 5% (w/w).

70. Composition according to claim 60, wherein the anti-microbial agent(s) is or comprises benzalkonium chloride; and wherein the one or more anti-microbial agent(s) is provided in a concentration of 0.01-2.5% (w/w), 0.025-1.0% (w/w), 0.05-0.5% (w/w), 0.075-0.2% (w/w), or around 0.1% (w/w).

71. Composition according to claim 60, wherein the one or more skin moisturizer(s)/skin conditioner(s) is or comprises panthenol; and wherein panthenol is provided in a concentration of 0.1-5% (w/w), 0.15-3.0% (w/w), 0.2-1.5% (w/w), 0.5-0.8% (w/w), or around 0.65% (w/w).

72. Composition according to claim 60, wherein the one or more skin moisturizer(s), skin conditioner(s), or skin moisturizer(s) and skin conditioner(s) is or comprises allantoin; and wherein allantoin is provided in a concentration of 0.01-5% (w/w), 0.05-2% (w/w), 0.1-1% (w/w), 0.2-0.5% (w/w), or around 0.3% (w/w).

73. A composition according to claim 60, comprising:
(I)
a. 20-85% (w/w), 40-75% (w/w), 50-70% (w/w), 55-65% (w/w), around 61% (w/w) water;
b. 0.1-60% (w/w), 5-55% (w/w), 10-50% (w/w), 30-50% (w/w), 35-45% (w/w), or around 30% (w/w) propylene glycol;
c. 0.1-15% (w/w), 0.5-12% (w/w), 1.0-10% (w/w), 1.5-7.5% (w/w), 2.0-6.0% (w/w), 2.5-4.0% (w/w), or around 5% (w/w) pentylene glycol;
d. 0.1-10% (w/w), 0.2-5% (w/w), 0.4-2.0% (w/w), 0.6-1.0% (w/w), or around 0.8% (w/w) gelator(s);
e. 0.1-5% (w/w), 0.15-2% (w/w), 0.2-1% (w/w), 0.25-0.75% (w/w), or around 0.5% (w/w) CBD;
f. 0.1-5% (w/w), 0.15-3.0% (w/w), 0.2-1.5% (w/w), 0.3-0.8% (w/w), or around 0.5% (w/w) panthenol;
g. 0.1-5% (w/w), 0.2-2% (w/w), 0.3-1% (w/w), 0.4-0.75% (w/w), or around 0.5% (w/w) hyaluronic acid, hyaluronate, or hyaluronic acid and hyaluronate;
h. 0.01-2.5% (w/w), 0.025-0.1.0% (w/w), 0.05-0.5% (w/w), 0.075-0.2% (w/w), or around 0.1% (w/w) benzalkonium chloride;
i. 0.01-5% (w/w), 0.05-2% (w/w), 0.1-1% (w/w), 0.2-0.5% (w/w), or around 0.3% (w/w) allantoin; and
j. 0.01-2.5% (w/w), 0.025-0.1.0% (w/w), 0.05-0.5% (w/w), 0.075-0.2% (w/w), or around 0.1% (w/w) phytic acid, phytate, or phytic acid and phytate;
or
(II)
a. 55-65% (w/w), around 61% (w/w) water
b. 35-45% (w/w), or around 30% (w/w) propylene glycol;
c. 2.5-4.0% (w/w), or around 5% (w/w) pentylene glycol;
d. 0.6-1.00% (w/w), or around 0.8% (w/w) gelator(s);
e. 0.25-0.75% (w/w), or around 0.5% (w/w) CBD;
f. 0.3-0.8% (w/w), or around 0.65% (w/w) panthenol;
g. 0.4-0.75% (w/w), or around 0.5% (w/w) hyaluronic acid, hyaluronate, or hyaluronic acid and hyaluronate;
h. 0.075-0.2% (w/w), or around 0.1% (w/w) benzalkonium chloride;
i. 0.2-0.5% (w/w), or around 0.3% (w/w) allantoin; and
j. 0.075-0.2% (w/w), or around 0.1 (w/w) phytic acid, phytate, or phytic acid and phytate.

74. A method for providing a wound treatment composition according to claim 60, comprising:
i. providing CBD in crystalline form and/or with a purity of at least 95% (w/w), 98% (w/w), 99% (w/w), 99.5% (w/w), or more than 99.8% (w/w);
ii. dissolving the CBD from step (i) in a penetrator enhancer;
iii. providing an aqueous gel;
iv. combining the dissolved CBD from step (ii) with the aqueous gel from step (iii); and
v. adding of the remaining ingredient(s) to provide the wound treatment composition.

75. A method for treating a wound of a subject comprising
applying a suitable amount of a composition according to claim 60 formulated as a topical preparation to a treatment area comprising the wound on the subject; and
providing a treatment to the wound,
wherein the wound is selected from one or more of:
a sore, clean wound, contaminated wound, infected wound, colonized wound; is related to a medical treatment, cosmetic treatment, surgery, tattooing, incision wound, laceration, abrasion, avulsion, puncture wound, gunshot wound, burn wound, first-degree burn, second-degree burn, third-degree burn, fourth degree burn, electromagnetic radiation-related burn, sunburn, UV light-related burn, frost bite, first-degree frostbite, second-degree frostbite, third-degree frostbite, fourth-degree frostbite, blister, chemical wound through contact with a chemical rash, allergy, wound related to a medical condition, by sore, bed sore, canker sore, cold sore, desert sore, pressure sore, saddle sore, summer sore, veldt sore, venereal sore, animal bite, an animal sting, or any combination thereof.

76. The method according to claim 75 wherein the suitable amount of the topical preparation for a single application comprises around 0.25 g of the topical preparation per 20-30 cm² treatment area on the subject; and
wherein 1, 2, 3, 4 or more than 4, applications of the topical preparation are applied to the treatment area on the subject per day.

77. The method of claim 76 wherein the around 0.25 g of the topical preparation comprises about a pea-sized volume of the topical preparation and is suitable for providing a thin film or layer of the topical preparation sufficient to cover the treatment area on the subject.

78. The method of claim 75 wherein the topical preparation is applied to the treatment area on the subject for a number of times sufficient to demonstrate healing of the wound.

79. A kit comprising an instruction for use and a receptacle comprising a wound treatment composition according to claim 60, said receptacle providing light and UV protection to the wound treatment composition
